(19) United States
(12) Patent Application Publication
(10) Pub. No.: US 2026/0345262 A1
(43) Pub. Date: Nov. 5, 2026

(54) INTERVENTION OPERATION ROBOT AND SYSTEM FOR INTERVENTION OPERATION CONTROL

(71) Applicant: SHANGHAI UNITED IMAGING HEALTHCARE CO., LTD., Shanghai (CN)

(72) Inventors: Meng LI, Shanghai (CN); Yanqi HUANG, Shanghai (CN); Zheng ZHANG, Shanghai (CN); Tuoyu CAO, Shanghai (CN)

(73) Assignee: SHANGHAI UNITED IMAGING HEALTHCARE CO., LTD., Shanghai (CN)

(21) Appl. No.: 19/714,862

(22) Filed: Jun. 22, 2026

Related U.S. Application Data
(63) Continuation of application No. PCT/CN2025/071249, filed on Jan. 8, 2025.

(30) Foreign Application Priority Data
Jan. 8, 2024 (CN) 202410021326.6

(51) Int. Cl. A61B 34/30; A61M 3/00; B25J 9/16

(52) U.S. Cl. CPC A61B 34/30; A61M 3/00; B25J 9/1679; B25J 9/1697

(57) ABSTRACT

The embodiments of the present disclosure provide an intervention operation robot and a system for intervention operation control. The system comprises: an injection auxiliary device configured to assist an injection instrument for injection; an intervention operation robot configured to convey an intervention instrument; an image acquisition device configured to acquire one or more images; and a control device configured to generate one or more control instructions based on feedback information from at least one of the injection auxiliary device, the intervention operation robot, and the image acquisition device, and send the one or more control instructions to at least one of the injection auxiliary device, the intervention operation robot, and the image acquisition device. The intervention operation robot includes an instrument conveying member and a robotic arm.

### CROSS-REFERENCE TO RELATED APPLICATIONS

This application is a Continuation of International Application No. PCT/CN2025/071249 filed on Jan. 8, 2025, which claims priority to Chinese Application No. 202410021326.6, filed on Jan. 8, 2024, the entire contents of which are incorporated herein by reference.

### TECHNICAL FIELD

The present disclosure relates to the technical field of equipment control for intervention operation, and in particular to an intervention operation robot and a system for intervention operation control.

### BACKGROUND

During a process of an intervention operation, a doctor needs to perform the operations for a long time in the presence of radiation, which causes harm to the doctor. In order to reduce radiation, the doctor may wear protective equipment such as a lead suit to operate. However, the heavy lead suit consumes a lot of physical strength of the doctor, and leads to a decrease in attention and stability, which easily affects the accuracy of the operation and increases the risk of surgery.

Therefore, an intervention operation robot and a system for intervention operation control are provided to simplify the process of intervention operation and improve the operation efficiency and operation accuracy with reduced manual intervention.

### SUMMARY

One or more embodiments of the present disclosure provide a system for intervention operation control. The system may comprise an injection auxiliary device configured to assist an injection instrument for injection; an intervention operation robot configured to convey an intervention instrument; an image acquisition device configured to acquire one or more images; and a control device configured to generate one or more control instructions based on feedback information from at least one of the injection auxiliary device, the intervention operation robot, or the image acquisition device, and send the one or more control instructions to at least one of the injection auxiliary device, the intervention operation robot, or the image acquisition device.

One or more embodiments of the present disclosure provide an intervention operation robot. The intervention operation robot may comprise an instrument conveying member configured to clamp at least one intervention instrument and convey the at least one intervention instrument to a target position of a subject; and a robotic arm detachably connected with the instrument conveying member and configured to control a movement of the instrument conveying member. The instrument conveying member may include one or more first convey assemblies. Each of the one or more first convey assemblies may be configured to clamp one of the at least one intervention instrument and drive the intervention instrument to translate and/or rotate.

One or more embodiments of the present disclosure provide an intervention instrument convey device configured to clamp at least one intervention instrument and convey the at least one intervention instrument to a target position of a subject. The intervention instrument convey device may comprise one or more first convey assemblies. Each of the one or more first convey assemblies may be configured to clamp one of at least one intervention instrument and drive the intervention instrument to move along an axial direction of the first convey assembly and/or drive the intervention instrument to rotate around an axis of the first convey assembly.

### BRIEF DESCRIPTION OF THE DRAWINGS

The present disclosure will be further illustrated by way of exemplary embodiments, which will be described in detail by means of the accompanying drawings. These embodiments are not limiting, and in these embodiments, the same numbering indicates the same structure, where:

FIG. 1 is a schematic diagram illustrating an application scenario of a system for intervention operation control according to some embodiments of present disclosure;

FIG. 2 is a schematic diagram illustrating an internal structure of a control device according to some embodiments of the present disclosure;

FIG. 3 is a block schematic diagram illustrating a control device according to some embodiments of the present disclosure;

FIG. 4 is a flowchart illustrating an exemplary method for intervention operation control according to some embodiments of the present disclosure;

FIG. 5 is a flowchart illustrating an exemplary process of determining first decision data according to some embodiments of the present disclosure;

FIG. 6 is a flowchart illustrating an exemplary process of determining a control instruction according to some embodiments of the present disclosure;

FIG. 7 is a flowchart illustrating an exemplary process of intervention operation control according to some embodiments of the present disclosure;

FIG. 8 is a schematic structural diagram illustrating an exemplary intervention operation robot according to some embodiments of the present disclosure;

FIG. 9 is a schematic structural diagram illustrating an exemplary instrument conveying member according to some embodiments of the present disclosure;

FIG. 10 is a schematic structural diagram illustrating another exemplary instrument conveying member according to some embodiments of the present disclosure;

FIG. 11 is a schematic structural diagram illustrating an exemplary support sleeve according to some embodiments of the present disclosure;

FIG. 12 is a schematic structural diagram illustrating an exemplary retractable base according to some embodiments of the present disclosure;

FIG. 13 is a schematic diagram illustrating an exploded structure of an instrument conveying member according to some embodiments of the present disclosure;

FIG. 14 is a schematic structural diagram illustrating an instrument conveying member according to some embodiments of the present disclosure;

FIG. 15 is a schematic diagram illustrating an exploded structure of a platform guide rail according to some embodiments of the present disclosure; and

FIG. 16 a schematic diagram illustrating an exploded structure of a support platform according to some embodiments of the present disclosure.

### DETAILED DESCRIPTION

In order to more clearly illustrate the technical solutions of the embodiments of the present disclosure, the accompanying drawings required to be used in the description of the embodiments are briefly described below. Obviously, the accompanying drawings in the following description are only some examples or embodiments of the present disclosure, and it is possible for a person of ordinary skill in the art to apply the present disclosure to other similar scenarios in accordance with these drawings without creative labor. Unless obviously obtained from the context or the context illustrates otherwise, the same numeral in the drawings refers to the same structure or operation.

Intervention surgery is a minimally invasive surgical technique that uses an intervention device such as a puncture needle, a catheter, and a guidewire to puncture into a human body to establish a treatment channel, transfer a specific intervention instrument such as a percutaneous balloon or a vascular stent to the lesion site of the human body, or inject a contrast agent, a chemotherapy drug, an embolic agent, etc., into the lesion site to diagnose and treat the local lesion under a guidance of an image acquisition device. The image acquisition device includes a digital subtraction angiography machine, a fluoroscopy machine, computed tomography (CT) equipment, magnetic resonance imaging (MRI) equipment, ultrasound (B-ultrasonography) equipment, etc.

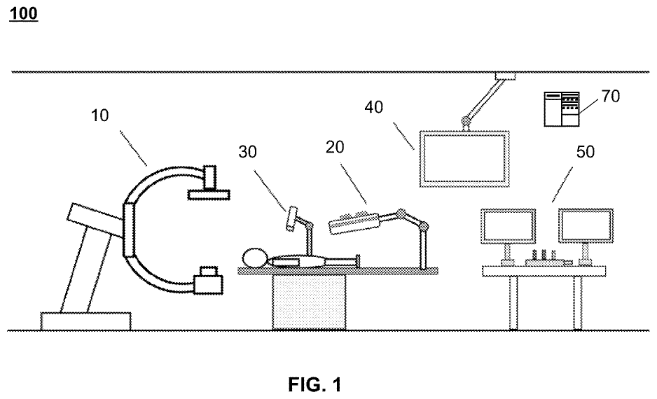

FIG. 1 is a schematic diagram illustrating an application scenario of a system for intervention operation control according to some embodiments of present disclosure.

In some embodiments, as shown in FIG. 1, a system for intervention operation control 100 may include an image acquisition device 10, an intervention operation robot 20, an injection auxiliary device 30, and a control device 70.

The image acquisition device 10 may be configured to acquire one or more images. For example, the image acquisition device may include at least one of an angiography device, a fluoroscopy device, a computed tomography (CT) device, a magnetic resonance imaging (MRI) device, an ultrasound (B-ultrasonography) device, etc. A C-arm x-ray device as shown in FIG. 1 is merely taken as an example of the image acquisition device and may not limit the scope of the present disclosure. The one or more images acquired by the image acquisition device 10 may include a preoperative image and an intraoperative image.

The intervention operation robot 20 may be configured to convey an intervention instrument. The intervention instrument refers to an instrument used in intervention instrument. For example, the intervention instrument may include a catheter, a guidewire, a puncture needle, a percutaneous balloon, an injection instrument, etc. The puncture needle, the catheter, the guidewire, etc., may enter a human body through puncture to establish a treatment channel which is used to transfer a specific intervention instrument such as a percutaneous balloon and a vascular stent to a lesion site of the human body.

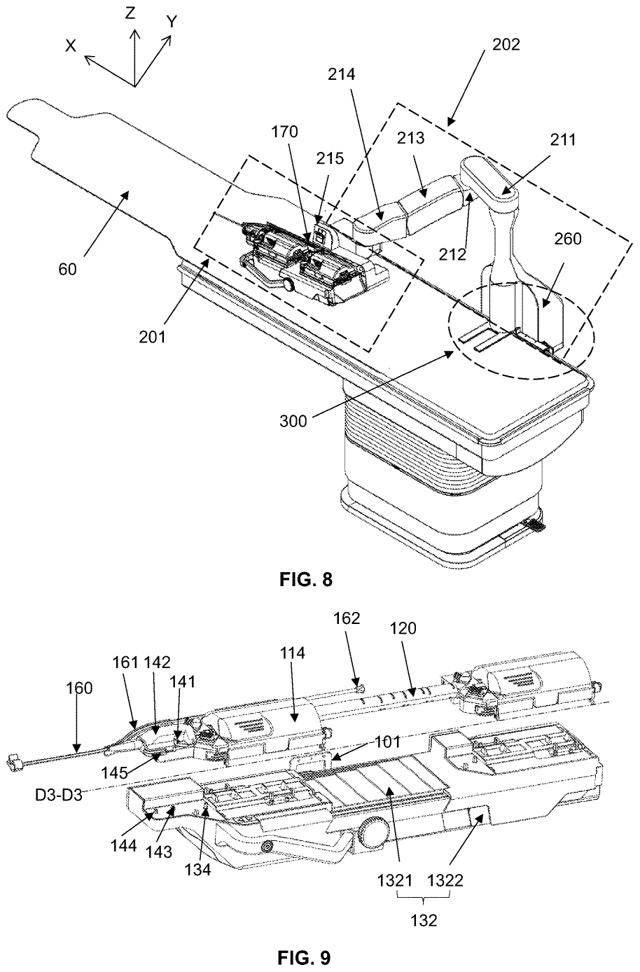

The intervention operation robot may include an instrument conveying member, a robotic arm, or the like. The instrument conveying member may clamp at least one of at least one intervention instrument and convey the intervention instrument to a target position (e.g., a target blood vessel or a lesion site) of a subject. More descriptions regarding the intervention operation robot may be found in FIGS. 8-15 and related descriptions thereof.

The injection auxiliary device 30 may be configured to assist an injection instrument for injection.

The injection instrument refers to a surgical instrument for performing an injection operation. For example, the injection instrument may include a syringe. The syringe may be connected the injection auxiliary device and removed when not in use. The model of the syringe may be selected as required.

The injection auxiliary device 30 may include a drive unit, a display unit, a detection unit, or the like. The drive unit may be configured to drive the injection instrument mounted thereon to move (e.g., move forward) to perform the injection operation. The display unit may be configured to display current injection information (e.g., a current injection speed, a current injection pressure, an injected dose, a remaining injection dose, etc.) and/or an intraoperative image. The intraoperative image may include an angiographic image (e.g., an angiographic image acquired by the image acquisition device 10) during an execution stage of the intervention operation, etc. The detection unit may be configured to detect injection information such as an injection pressure, an injection dose and flow rate, whether the injection instrument is placed correctly, an instruction associated with the injection operation input by a doctor.

The injection auxiliary device 30 may adjust an injection parameter based on one or more control instructions to control the injection instrument to complete automatic injection for a subject. For example, the injection auxiliary device 30 may automatically adjust the injection parameter based on a received second control instruction in combination with the detected current injection speed and the current injection pressure. The second control instruction may include information such as a target injection speed, a target injection pressure, a target injection dose, or the like, or a combination thereof. For example, if the current injection speed reaches the target injection speed, the current injection pressure may be maintained until the injection dose reaches the target injection dose; if the current injection speed does not reach the target injection speed and the current injection pressure does not reach an injection pressure limit (also referred to as an injection pressure upper limit), the injection auxiliary device 30 may increase the current injection pressure until the current injection speed reaches the target injection speed or the injection dose reaches the target injection dose.

The control device 70 may be configured to generate one or more control instructions based on feedback information and/or data from at least one of the image acquisition device 10, the intervention operation robot 20, and the injection auxiliary device 30, and send the one or more control instructions to at least one of the image acquisition device 10, the intervention operation robot 20 and the injection auxiliary device 30.

The feedback information reflects a current parameter setting, an operation state, a fault state, etc., of the injection auxiliary device, the intervention operation robot, and/or image acquisition device. For example, the feedback information of the injection auxiliary device 30 may include data such as the current injection dose, the current injection speed, and the current injection pressure. The feedback information of the injection auxiliary device 30 may reflect a current state of the injection instrument. As another example, the feedback information of the intervention operation robot 20 may include data such as a current convey distance, a current rotation angle, a current convey speed, or the like. The feedback information of the intervention operation robot may reflect a current state of the intervention instrument, such as force information and movement information of the intervention instrument. As another example, the feedback information of the image acquisition device may include image data such as the preoperative image and the intraoperative image acquired by the image acquisition device 10, and/or an inspection parameter (e.g., a scanning parameter such as a ray emission time and emission amount). The intraoperative image may be a contrast agent vascular image or a non-contrast agent vascular image. The image data fed back by the image acquisition device may reflect an anatomical position represented in the intraoperative image, a vascular morphology, a position, a size and morphology of the intervention instrument, an image quality, stage information of the intervention operation, etc.

The image data may include at least one of the contrast agent vascular image, the non-contrast agent vascular image, or an image containing the intervention instrument. The image data may be at least one of a two-dimensional (2D) image or a three-dimensional (3D) image. The 3D image is usually acquired before the intervention operation. The 2D image may be acquired during surgery through digital subtraction angiography (DSA).

In some embodiments, the feedback information (also referred to as first sensor data) of the intervention operation robot 20 may be obtained through one or more sensors. For example, the intervention operation robot 20 may obtain the current convey distance, the current rotation angle, the current convey speed, or the like, through the one or more sensors disposed on the intervention operation robot 20; or directly measure the force information and the movement information of the intervention instrument clamped by the intervention operation robot 20 through the one or more sensors.

In some embodiments, the feedback information (also referred to as second sensor data) of the injection auxiliary device 30 may be obtained through one or more sensors. For example, the injection auxiliary device 30 may measure and obtain the current injection speed, the current injection pressure, or the like, through the one or more sensors disposed on the injection auxiliary device 30.

The one or more control instructions are instruction information for controlling the operation of the injection auxiliary device, the intervention operation robot, and/or the image acquisition device. In some embodiments, the one or more control instructions may include a first control instruction, a second control instruction, and a third control instruction. The first control instruction is an instruction related to the image acquisition device 10. For example, the first control instruction may indicate whether to acquire an image, whether to adjust an acquisition posture, a specific acquisition parameter, or the like. The acquisition parameter may include an acquisition protocol, an acquisition frequency, the acquisition posture, an acquisition duration, etc. The second control instruction is an instruction related to the injection auxiliary device 30. For example, the second control instruction may indicate whether to inject a drug, a target injection dose, a target injection speed, an injection pressure upper limit, etc. The third control instruction is an instruction related to the intervention operation robot. For example, the third control instruction may indicate a type of the intervention instrument conveyed by the intervention operation robot, a convey distance, a rotation angle, a convey speed, etc.

The control device 70 may be configured to generate the one or more control instructions based on the feedback information in various ways.

For example, the control device 70 may obtain the injection information (e.g., the current injection speed, the current injection pressure, the current injection dose, etc.) fed back by the injection auxiliary device 30, and determine whether the injection information satisfies a preset injection condition; and in response to determining that the injection information does not satisfy the preset injection condition, generate the second control instruction related to the injection auxiliary device 30 to control the injection auxiliary device 30 to adjust the injection parameter. For example, the preset injection condition may include that the injection dose reaches the target injection dose. In response to determining that the current injection volume is less than the target injection dose, the control device may generate the second control instruction related to the injection auxiliary device 30 to make the injection auxiliary device 30 perform injection control based on the second control instruction (e.g., the injection auxiliary device 30 increases the injection pressure) until the current injection dose reaches the target injection dose. As another example, the control device 70 may determine the target injection dose and a blood flow rate of a blood vessel where a catheter tip is located based on an angiographic image fed back by the image acquisition device 10, obtain the target injection speed based on the blood flow rate, and obtain the injection pressure limit based on a catheter parameter (e.g., a catheter length, a catheter type, the injection pressure limit of the catheter, etc.), generate the second control instruction based on the target injection dose, the target injection speed, and the injection pressure limit, and send the second control instruction to the injection auxiliary device 30 to make the injection auxiliary device 30 adjust the one or more injection parameters based on the second control instruction.

As another example, the control device 70 may determine the one or more control instructions related to at least one of the injection auxiliary device, the intervention operation robot, or the image acquisition device based on first image data acquired by the image acquisition device 10. The first image data may include the preoperative image and the intraoperative image acquired by the image acquisition device 10 in a current acquisition posture. For example, the control device 70 may determine an initial vascular diagram based on the preoperative image; determine an initial acquisition posture and an initial acquisition protocol of the image acquisition device based on the initial vascular diagram and a target blood vessel; and determine the first control instruction related to the image acquisition device 10 based on the initial acquisition posture and the initial acquisition protocol. The first control instruction is used to cause the image acquisition device to perform parameter setting based on the initial acquisition posture and the initial acquisition protocol. The initial vascular diagram refers to a 2D or 3D vascular diagram acquired before surgery. The initial vascular diagram may include spatial position information of blood vessels within an expected scope of surgery. The initial vascular diagram may further include a safety region, i.e., a vascular region where the intervention instrument can travel.

As another example, the control device 70 may determine a reference acquisition posture by aligning the first image data with a plurality of first reference image data; and determine the first control instruction based on the current acquisition posture and the reference acquisition posture of the image acquisition device corresponding to the first image data, the first control instruction being used to adjust the image acquisition device from the current acquisition posture to the reference acquisition posture. A first reference image data refers to a 3D preoperative or intraoperative image acquired in advance. In this case, the first image data is a 2D image. The reference acquisition posture refers to an acquisition posture that minimizes a projection overlap of 2D image data. The control device 70 may align the first image data with each of the plurality of first reference image by registering the first image data with each of the plurality of first reference image data. Each of the plurality of first reference image data may correspond to a first reference acquisition posture at which the first reference image data is acquired. The control device 70 may determine an overlapping area between the first image data and each of the plurality of first reference image data. The control device 70 may determine one of the plurality of first reference image data with a minimum overlapping area with the first image data and designate the first reference acquisition posture of the one of the plurality of first reference image data with a minimum overlapping area with the first image data as the reference acquisition posture of the first image data. The control device may compare the reference acquisition posture with the current posture of the image acquisition device to generate the first control instruction, and adjust the image acquisition device to the reference acquisition posture.

As another example, in response to determining that the image acquisition device 10 is adjusted from the current acquisition posture to the reference acquisition posture, the control device 70 may determine the second control instruction, and feed the second control instruction back to the injection auxiliary device 30. The second control instruction is used to cause the injection auxiliary device 30 to control the injection device to perform injection. In response to determining that the injection instrument completes injection, the control device 70 may update the initial vascular diagram based on the second image data acquired by the image acquisition device in the reference acquisition posture to obtain an updated vascular diagram. The second image data refers to a preoperative image and an intraoperative image that are re-acquired after the current acquisition posture of the image acquisition device is adjusted to the reference acquisition posture.

As another example, the control device 70 may determine the state information of the intervention instrument clamped by the intervention operation robot 20 based on the second image data; and determine the third control instruction based on the updated vascular diagram, the state information of the intervention instrument, the force information of the intervention instrument, and the movement information of the intervention instrument gripped by the intervention operation robot. The third control instruction is used to cause the intervention operation robot 20 to convey the intervention instrument to a target region (e.g., the target blood vessel or the lesion site of the subject). The force information reflects a force condition and a force change during the operation of the intervention instrument. The force information may include a magnitude of the force, a force change etc. The movement information may include speed, acceleration, and other information of the intervention instrument during operation, such as a convey speed, a convey acceleration, etc. More descriptions regarding the state information of the intervention instrument may be found in FIG. 5 and related descriptions thereof.

In some embodiments, as shown in FIG. 1, the system for intervention operation control 100 may further include a display device 40 and a terminal device 50. The display device 40 may be configured to display information related to the system for intervention operation control 100. For example, the control device 70 may send the obtained feedback information (e.g., the image data, the acquisition data, etc., fed back by the image acquisition device 10, the current convey distance, the current rotation angle, the current convey speed, etc., fed back by the intervention operation robot 20, and the current injection speed, the current injection pressure, etc., fed back by the injection auxiliary device 30) to the display device 40 for a display to a user. As another example, the control device 70 may send the state information (e.g., the force information, the movement information, morphology information, curvature information, position information, or the like, of the intervention instrument, relevant information of the injection instrument, the current stage of the intervention operation) and/or decision data (e.g., first decision data), etc., obtained based on the feedback information to the display device 40 for display.

The terminal device 50 is a device for performing an operation by an operator (e.g., a doctor). The doctor may view the relevant information of the system for intervention operation control 100 through the display device 40 or the terminal device 50, and perform an operation such as information confirmation or selection through the terminal device 50. For example, the user may input second decision data through the terminal device 50 to confirm the first decision data and/or the state information. The terminal device may be a computer, a mobile terminal, etc.

According to some embodiments of the present disclosure, various modules of the system for intervention operation control 100 (e.g., the image acquisition device 10, the intervention operation robot 20, and the injection auxiliary device 30, etc.) can be collaboratively controlled without the involvement of the doctor, and the processes of conveying the intervention instrument to an appropriate position, setting the acquisition parameter, acquiring the image data, and injecting a drug such as a contrast agent can be automatically completed, thereby simplifying the process of the intervention operation, and improving surgical efficiency.

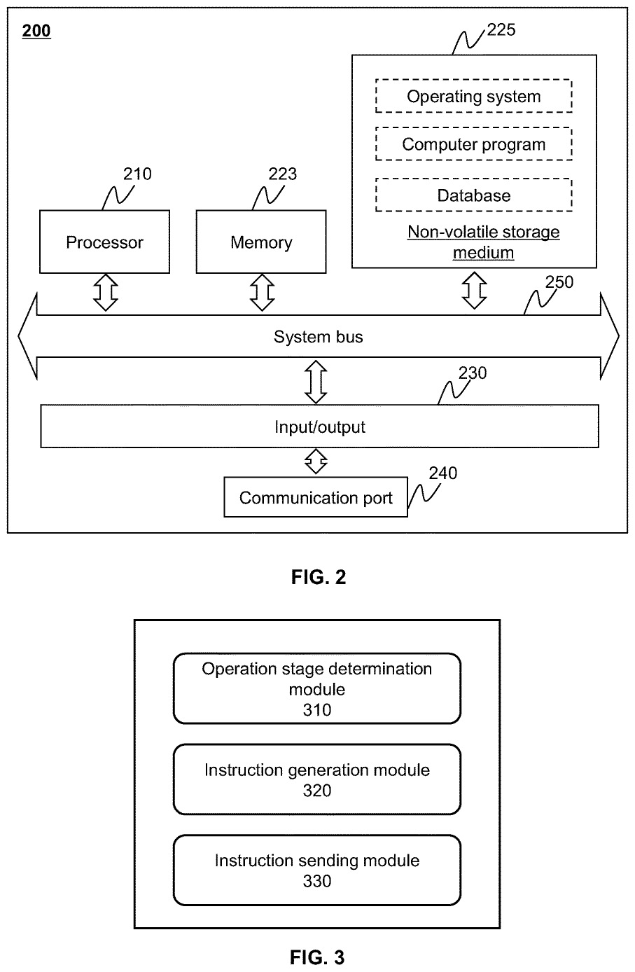

FIG. 2 is a schematic diagram illustrating an internal structure of a control device according to some embodiments of the present disclosure.

In some embodiments, the control device 70 may be implemented by a computing device 200. In some embodiments, the computing device 200 may include a server, a personal computer, a laptop computer, a smart phone, a tablet computer, a smart mobile phone, etc.

As shown in FIG. 2, the computing device 200 may include a processor 210, a storage device, an input/output 230, and a communication port 240. The storage device may include a non-volatile storage medium 225 and a memory 223. The processor 210, storage device (e.g., the memory 223, and the non-volatile storage medium 225), and the input/output 230 may be connected via a system bus 250. The communication port 240 may be connected to the system bus 250 via the input/output 230.

The processor 210 may execute computer instructions (e.g., a program code) and may perform functions of a processing device in accordance with the techniques described in the present disclosure. In some embodiments, the processor 210 may include one or more hardware processors, such as a microcontroller, microprocessor, etc. For illustrative purposes only, only one processor is described in computing device 200. However, it is noted that the computing device 200 may also include a plurality of processors. Operations and/or methods described in the present disclosure that are performed by a single processor may also be performed by a plurality of processors together or separately. For example, if a processor of the computing device 200 described in the present disclosure performs an operation A and an operation B, it should be appreciated that the operation A and the operation B may also be performed by two or more different processors of the computing device 200 jointly or separately (e.g., a first processor executing the operation A and a second processor executes the operation B, or the first processor and the second processor jointly execute the operations A and B).

The storage device may store data/information obtained from the image acquisition device 10, the intervention operation robot 20, the injection auxiliary device 30, and/or any other component of the system 100. For example, the non-volatile storage medium 225 may store an operating system, a computer program and a database. The memory 223 may provide an environment for an operation of the operating system and computer programs in the non-volatile storage medium 225. The database may be configured to store data during intervention operation control (e.g., a preoperative image and/or an intraoperative image acquired by the image acquisition device 10, a current convey distance, a current rotation angle, a current convey speed, and other information fed back by the intervention operation robot 20, a current injection dose, a current injection speed, a current injection pressure, and other information fed back by the injection auxiliary device 30). The processor 210 may execute the computer programs to implement a method for intervention operation control described herein.

The input/output 230 may be configured to exchange information between the processor 210 and an external device, such as the image acquisition device 10, the intervention operation robot 20, and the injection auxiliary device 30. In some embodiments, the input/outputs 230 may include an input device and an output device. The input device may include a keyboard, a mouse, a touch screen, a microphone, etc., or any combination thereof. The output device may include a display device, a speaker, a printer, a projector, or the like, or any combination thereof.

The communication port 240 may be configured to communicate with an external terminal (e.g., the image acquisition device 10, the intervention operation robot 20, and the injection auxiliary device 30) via a network connection. The connection may be a wired connection, a wireless connection, any connection that enables data transmission and/or reception, or the like, or any combination thereof.

It should be understood that the descriptions of FIGS. 1-2 are only provided for the purpose of illustration and do not constitute a limitation to the present disclosure. For those skilled in the art, various changes and modifications can be made under the guidance of the present disclosure. Features, structures, manners, and other characteristics of embodiments of the present disclosure can be combined in various ways to obtain other and/or alternative embodiments. However, such changes and modifications do not exceed the scope of the present disclosure.

FIG. 3 is a block schematic diagram illustrating a control device according to some embodiments of the present disclosure.

As shown in FIG. 3, in some embodiments, the control device 70 may include an operation stage determination module 310, an instruction generation module 320, and an instruction sending module 330. In some embodiments, one or more modules of the control device 70 may be connected with each other. The connection may be a wireless connection or a wired connection.

The surgery stage determination module 310 may be configured to determine a stage of an intervention operation being performed based on feedback information.

The instruction generation module 320 may be configured to determine one or more control instructions that match the next stage of the stage of the intervention operation being performed.

The instruction sending module 330 may be configured to send the one or more control instructions to at least one of the injection auxiliary device, the intervention operation robot, and the image acquisition device.

In some embodiments, the instruction generation module 320 may be further configured to: determine, based on the feedback information, first decision data that matches the next stage of the stage of the intervention operation being performed; and generate the one or more control instructions based on the first decision data in response to receiving a confirmation operation of a user on the feedback information and the first decision data.

In some embodiment, the instruction generation module 320 may be further configured to: determine first state information of the intervention operation robot based on the feedback information; determine second state information of the intervention instrument clamped by the intervention operation robot based on the first state information of the intervention operation robot and an intraoperative image; and determine, based on the first state information of the intervention operation robot and the second state information of the intervention instrument, the first decision data matching the next stage of the stage of the intervention operation being performed.

In some embodiments, the instruction generation module 320 may be further configured to: compare the intraoperative image with a reference image; generate, based on a comparison result, a target acquisition protocol and a target acquisition posture of the image acquisition device; and generate a control instruction associated with the image acquisition device based on the target acquisition protocol and the target acquisition posture, the control instruction being used to cause the image acquisition device to perform a parameter setting based on the target acquisition protocol and the target acquisition posture.

In some embodiment, the operation stage determination module 310 may be further configured to: determine a feature analysis result by analyzing the image acquired by the image acquisition device during the intervention operation; and determine, based on the feature analysis result, the stage of the intervention operation being performed.

In some embodiments, the control device 70 may further include a manual decision processing module. The manual decision processing module may be configured to: during an execution of a corresponding control instruction by one of the injection auxiliary device, the intervention operation robot, and the image acquisition device, control a device associated with second decision data to stop executing a control instruction if the second decision data inputted by a user is received; and generate a decision instruction based on the second decision data. The instruction sending module 330 may be further configured to: send the decision instruction to the device associated with the second decision data.

The modules of the system for intervention operation control can be implemented in whole or in part by software, hardware, or a combination thereof. The modules can be embedded in or independent of the processor of the control device in the form of hardware, or can be stored in the memory of the control device in the form of software, such that the processor can call and execute the operations corresponding to the modules, respectively.

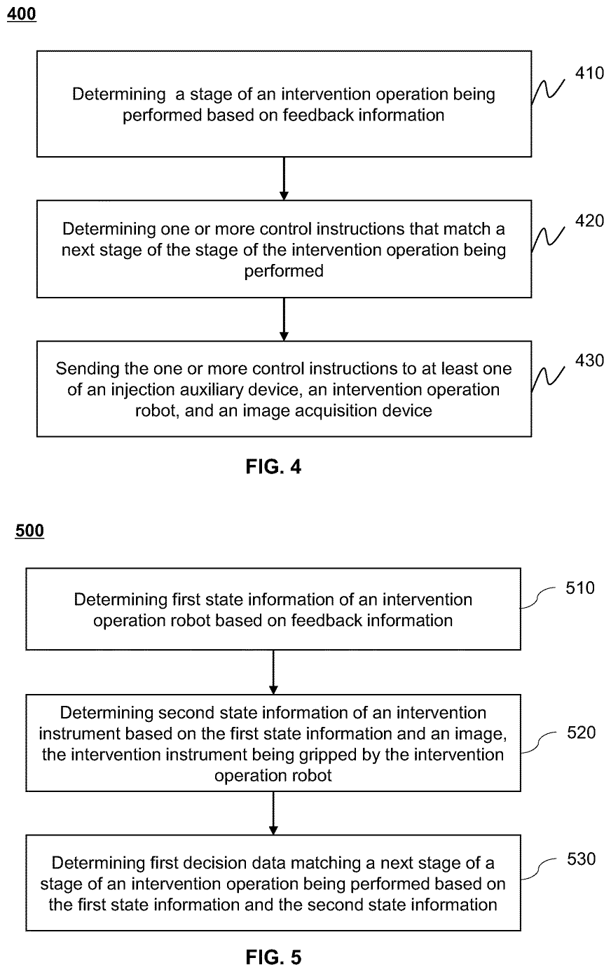

FIG. 4 is a flowchart illustrating an exemplary process for intervention operation control according to some embodiments of the present disclosure.

In some embodiments, the intervention operation may include a plurality of stages in chronological order. For example, in the chronological order, the intervention operation may include a plurality of stages such as preoperative planning, selective vascular cannulation, angiography, superselective vascular cannulation, intervention instrument conveying, drug injection, instrument withdrawal, etc.

In some embodiments, generating one or more control instructions based on feedback information from at least one of the injection auxiliary device, the intervention operation robot, or the image acquisition device may include: determining, based on the feedback information, a stage of the intervention operation being performed; and determining the one or more control instructions that match a next stage of the stage of the intervention operation being performed. More descriptions regarding determining the one or more control instructions that match the next stage of the stage may be found in the related descriptions of FIG. 4.

In some implementations, as shown in FIG. 4, a process 400 is a flowchart illustrating an exemplary process for intervention operation control. The process 400 may include operations 410-430. The operations 410-430 may be executed by the control device 70.

In 410, a stage of an intervention operation being performed may be determined based on feedback information.

In some embodiments, during the intervention operation, after the image acquisition device acquires a current intraoperative image, the current intraoperative image may be fed back to the control device. The control device may determine the stage of the intervention operation being performed by analyzing the current intraoperative image.

In some embodiments, if the feedback information includes an image acquired by the image acquisition device during the intervention operation, the control device 70 may determine a feature analysis result by analyzing the image acquired by the image acquisition device 10 during the intervention operation; and determine, based on the feature analysis result, the stage of the intervention operation being performed.

Feature analysis may use an edge detection manner, or other image detection manners. A feature may include information related to an anatomical position carried by the image, a vascular morphology, a position, a size, a morphology of the intervention instrument, an image quality, etc.

In some embodiments, the control device may perform feature analysis on the image acquired by the image acquisition device during the intervention operation through a neural network model, and predict, based on the feature analysis result, the stage of the intervention operation being performed, and/or the next stage of the stage of the intervention operation being performed.

By determining the stage of the intervention operation being performed through feature analysis, the determination result can be more accurate, thereby making the control instructions for the next stage of the operation generated subsequently more accurate.

In 420, one or more control instructions that match the next stage of the stage of the intervention operation being performed may be determined.

In some embodiments, after determining the stage of the intervention operation being performed, the control device may determine, based on the plurality of stages and the chronological order thereof, a stage that is adjacent to and after the stage of the intervention operation being performed as the next stage.

In some embodiments, when the intervention operation is in different stages or the intervention instrument is in different operation states, there are different requirements for the frequency and duration of image acquisition. To this end, a control strategy of the image acquisition device may be dynamically adjusted according to the stage of the intervention operation or the state information of the intervention instrument.

In some embodiments, the control device may determine the one or more control instructions that match the next stage of the stage of the intervention operation being performed in various ways. For example, in response to determining that the control device 70 determines that the current operation of the intervention instrument may have a risk based on the feedback information (e.g., the image data fed back by the image acquisition device and the first sensor data fed back by the intervention operation robot), the control device 70 may change the control strategy of the image acquisition device 10. For example, the frequency and the duration of image acquisition may be increased to increase the input of image information acquired by the image acquisition device, so as to accurately determine the current risk. For example, when a catheter or a guidewire is located in or near a blood vessel or a location without bifurcation, the intervention operation is in a relatively low risk stage, and a first control instruction for reducing the frequency of image acquisition is generated; when the catheter or the guidewire enters a region where the blood vessels are dense in the next stage, a first control instruction for reducing the convey speed of the intervention operation robot, and increasing the acquisition frequency and the acquisition duration of the image acquisition device is generated.

As another example, the control device 70 may analyze the image fed back by the image acquisition device 10 to determine that the intervention instrument reaches a target blood vessel, and determine that the stage of the intervention operation being performed is conveying the intervention instrument, and the next stage is drug injection. The control device 70 may generate a first control instruction for adjusting an acquisition parameter of the image acquisition device 10, and generate a second control instruction for controlling an injection operation of the injection auxiliary device.

In some embodiments, during the process of conveying the intervention instrument by the intervention operation robot, there is a certain amount of information loss in the information in a real-time 2D image compared with the information in a real 3D movement. As the intervention instrument continues to move, the intervention instrument may be blocked and overlapped in the 2D image. In this case, position information of the intervention instrument obtained through the image data and movement information of the intervention instrument obtained through the sensor data may be mismatch, which causes a decrease in the confidence of the control instruction. Therefore, the control device may adjust the acquisition posture of the image acquisition device based on the feedback information such that the image acquisition device can acquire image data from multiple angles to compensate for the information loss.

In some embodiments, the control device may intelligently generate an image acquisition strategy after determining the stage of the intervention operation being performed based on the image data fed back by the image acquisition device. For example, with the doctor's permission, the control device may determine one or more control instructions related to the on-off of DSA rays, a contrast agent injection amount and time, an MR sequence type and scanning time, an ultrasound acquisition time, or the like, so as to acquire one or more images when necessary, thereby saving operation time, and reducing unnecessary radiation and contrast agent injection.

In some embodiments, generating one or more control instructions based on feedback information from at least one of the injection auxiliary device, the intervention operation robot, or the image acquisition device may include: determining, based on the feedback information, first decision data matching a next stage of the stage of the intervention operation being performed; and generating the one or more control instructions based on the first decision data in response to receiving a confirmation operation on the feedback information and the first decision data.

The first decision data refers to data automatically generated by the control device and related to the next stage of the intervention operation. In some embodiments, the control device may analyze and sort out the data related to the next stage of the intervention operation as the first decision data based on the feedback information from at least one of the image acquisition device, the injection auxiliary device, or the intervention operation robot.

In some embodiments, after determining the stage of the intervention operation being performed, the control device may determine the first decision data related to the image acquisition device, such as an acquisition parameter of the image acquisition device.

In some embodiments, after determining the stage of the intervention operation being performed, the control device may determine the first decision data related to the injection auxiliary device. For example, the control device 70 may determine an injection speed adjustment change, an injection pressure adjustment change, an injection dose adjustment change, or the like, or a combination thereof based on a current injection speed, a current injection pressure, a current injection dose and other information fed back by the injection auxiliary device 30; or determine a target injection speed, an injection pressure upper limit, a target injection dose, or the like, or a combination thereof based on the image fed back by the image acquisition device.

In some embodiments, after determining the stage of the intervention operation being performed, the control device may determine the first decision data related to the intervention operation robot. More descriptions regarding determining the first decision data related to the intervention operation robot may be found in FIG. 5 and related descriptions thereof.

The next stage may be a different stage of the intervention operation. For example, the next stage of an injection stage may be a continued input stage of the intervention instrument, etc.

In some embodiments, after obtaining the feedback information, the control device may display the feedback information to an operator (e.g., a doctor) through a display device (e.g., display device 40) for user confirmation. The control device may also display the first decision data to the doctor through the display device for user confirmation.

The manner of generating the one or more control instructions based on the first decision data may be similar to the manner of generating the control instruction based on the feedback information. More descriptions may be found in the related descriptions of FIG. 1.

In some embodiments, the control device may determine the one or more control instructions through an instruction determination model based on the image data acquired by the image acquisition device 10 and/or the first sensor data and the second sensor data.

The instruction determination model is a model for determining the one or more control instructions. In some embodiments, the instruction determination model may be a machine learning model. For example, the instruction determination model may include at least one of a convolutional neural network (CNN) model, a recurrent neural network (RNN) model, or other models. An input of the instruction determination model may include the image data, the first sensor data, and/or the second sensor data, and an output of the instruction determination model may include the one or more control instructions.

In some embodiments, the instruction determination model may be obtained by training based on training samples and training labels corresponding to the training samples. In some embodiments, each of a plurality of training samples with training labels may be input into an initial instruction determination model, and a loss function may be constructed from the training label and a result of the initial instruction determination model corresponding to each of the plurality of training samples. Parameters of the initial instruction determination model may be iteratively updated based on the loss function by gradient descent or other methods. When a preset condition is met, the model training is completed and a trained instruction determination model is obtained. The preset condition may be that the loss function converges, a count of iterations reaches a threshold, etc.

Each of the training samples may include at least one of sample image data, sample first sensor data and/or sample second sensor data in sample data. The training samples may be obtained through historical data. The historical data may include historical image data, historical first sensor data, and historical second sensor data. The training labels corresponding to the training samples may include one or more sample control instructions corresponding to each of the training samples. The training labels may be obtained through manual annotation. For example, the training labels may include manually input adjustment information for parameters of the image acquisition device 10, the intervention operation robot 20, the injection auxiliary device 30, etc.

In some embodiments, during an execution of a corresponding control instruction by one of the injection auxiliary device, the intervention operation robot, and the image acquisition device, a target device associated with the second decision data may be controlled to stop executing a control instruction if the second decision data input by a user is received; and a decision instruction is generated based on the second decision data and sent to the target device associated with the second decision data. The target device may be any one of the injection auxiliary device, the intervention operation robot, and the image acquisition device.

The second decision data refers to data input by the user for generating a control instruction. The decision instruction is an instruction generated based on the second decision data.

The second decision instruction is generated through the second decision data to ensure that the user has the highest priority and realizes the manual decision. When the user finds a risk or has a better decision, the execution of the corresponding control instruction by one of the injection auxiliary device, the intervention operation robot, and the image acquisition device can be stopped in time, or the device can execute the better decision of the user, thereby improving the reliability and safety of the operation.

In 430, the one or more control instructions may be sent to at least one of an injection auxiliary device, an intervention operation robot, and an image acquisition device.

At least one of the injection auxiliary device, the intervention operation robot, and the image acquisition device may perform an operation based on the corresponding control instruction.

By generating the one or more control instructions for the next stage by determining the current stage of the operation, parameters of various components of the system for intervention operation control system can be adjusted in advance, thereby preventing possible risks during the operation, and making the operation process safer.

FIG. 5 is a flowchart illustrating an exemplary process of determining first decision data according to some embodiments of the present disclosure.

In some embodiments, feedback information may include an image acquired by an image acquisition device during an intervention operation. The control device 70 may determine first state information of an intervention operation robot based on the feedback information; determine, based on the first state information and the image, second state information of the intervention instrument, the intervention instrument being gripped by the intervention operation robot; and determine, based on the first state information and the second state information, the first decision data matching the next stage of the stage of the intervention operation being performed.

In some implementations, as shown in FIG. 5, a process 500 is flowchart illustrating an exemplary process of determining the first decision data. The process 500 may include operations 510-530. The operations 510-530 may be performed by the control device 70.

In 510, first state information of an intervention operation robot may be determined based on feedback information.

The intervention operation robot may be configured to clamp an intervention instrument to convey the intervention instrument to a target position. The first state information of the intervention operation robot reflects a current state of the intervention instrument gripped by the intervention operation robot, such as force information and movement information of the intervention instrument. The force information of the intervention instrument may include a force magnitude of the intervention instrument, a force change, a force change mode, a force distribution in space, a force change trend within a time period, and other information.

The control device may determine the first state information of the intervention operation robot by obtaining the target information of the intervention operation robot based on the feedback information from the image acquisition device, the injection auxiliary device, and the intervention operation robot. For example, after the control device 70 receives the feedback information sent by the image acquisition device 10, the intervention operation robot 20, and the injection auxiliary device 30, the control device 70 may obtain the target information sent by the intervention operation robot 20, and determine the force information (e.g., a force magnitude) and the movement information (e.g., a movement distance, a movement angle, etc.) of the intervention instrument based on the target information sent by the intervention operation robot 20.

In 520, second state information of an intervention instrument may be determined based on the first state information and an image, the intervention instrument being gripped by the intervention operation robot.

The second state information reflects the state and position information of the intervention instrument. For example, the second state information may include a morphology and a tortuosity of the intervention instrument. As another example, the second state information may include a spatial position of the intervention instrument, or a position in a blood vessel, etc. In some embodiments, the control device may determine the second state information based on the first state information and the image in various ways. For example, the control device 70 may determine the second state information based on the first state information and the image acquired by the image acquisition device during the intervention operation in various ways (e.g., using a trained machine learning model, statistical analysis, etc.).

In 530, first decision data matching a next stage of a stage of an intervention operation being performed may be determined based on the first state information and the second state information.

In some embodiments, the control device may determine the first decision data of the next stage of the stage of the intervention operation being performed based on the first state information and the second state information in various ways. For example, the control device may determine whether the intervention instrument is in a safety region based on the first state information and the second state information, and generate a first control instruction for adjusting a parameter of the image acquisition device 10 or a third control instruction for adjusting a parameter of the intervention operation robot as the first decision data in response to determining that the intervention instrument is not in the safety region. For example, the control device may determine a convey distance, a rotation angle, a convey speed, or the like, of the intervention operation robot based on the first state information and the second state information of the intervention instrument, so as to convey the intervention instrument to a target position or a target vessel.

In some embodiments, the control device may display current state data (e.g., the feedback information of the image acquisition device, the injection auxiliary device, and the intervention operation robot) of each device of the system to the user, and generate the first decision data matching the next stage of the stage of the intervention operation being performed based on the first state information of the intervention operation robot and the second state information of the intervention instrument in response to a confirmation operation of the user.

The acquisition parameter is dynamically adjusted based on the state of the system for intervention operation control and the intervention instrument, so as to avoid occlusion and overlap of the intervention instrument in the image.

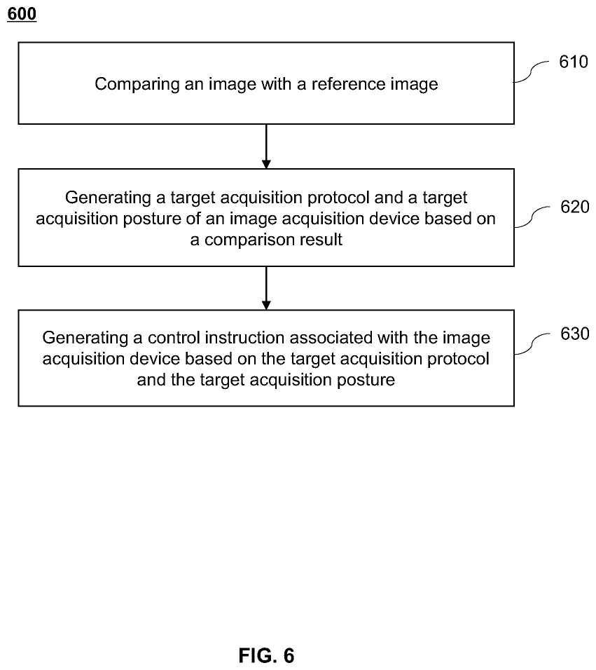

FIG. 6 is a flowchart illustrating an exemplary process of determining a control instruction according to some embodiments of the present disclosure. A process 600 may be executed by the control device 70.

In some embodiments, generating one or more control instructions based on feedback information from at least one of an injection auxiliary device, an intervention operation robot, or an image acquisition device may include: comparing an image with a reference image, the image being acquired by the image acquisition device during an intervention operation; generating, based on a comparison result, a target acquisition protocol and a target acquisition posture of the image acquisition device; and generating a control instruction associated with the image acquisition device based on the target acquisition protocol and the target acquisition posture, the control instruction being used to cause the image acquisition device to perform a parameter setting based on the target acquisition protocol and the target acquisition posture.

In some implementations, as shown in FIG. 6, the process 600 is a flowchart illustrating an exemplary process of determining a control instruction of an image acquisition device. The process 600 may include operations 610-630.

In 610, an image may be compared with a reference image.

The reference image is a standard image used for reference. The reference image may be determined based on historical images (e.g., a preoperative image, and preoperative or intraoperative images acquired during previous operations) acquired by the image acquisition device 10. For example, for different lesion sites, an image with the clearest image or the clearest image of a lesion site in the historical images may be selected as the standard image (i.e., the reference image); or a preoperative or intraoperative 3D image may be selected as the reference image.

In some embodiments, the control device may compare the image and the reference image by comparing an image parameter of the image with an image parameter of the reference image. The image parameter may include an image signal intensity, a signal-to-noise ratio, an acquisition frame rate, etc.

In 620, a target acquisition protocol and a target acquisition posture of an image acquisition device may be generated based on a comparison result.

In some embodiments, the control device may generate the target acquisition protocol and the target acquisition posture of the image acquisition device in response to determining that the image parameter of the image does not satisfy a preset condition. Whether the image parameter of the image satisfies the preset condition may be determined by determining whether the comparison result between the image parameter of the image and the image parameter of the reference image satisfies the preset condition. The preset condition may be set by those skilled in the art based on experience. For example, the preset condition may be that a difference between the image parameter of the image and the image parameter of the reference image is less than a difference threshold, or a ratio of the image parameter of the image to the image parameter of the reference image is less than a ratio threshold. In response to determining that the image parameter does not satisfy the preset condition, the control device may determine an acquisition protocol and an acquisition posture of the reference image as the target acquisition protocol and target acquisition posture.

In some embodiments, the control device may determine a reference acquisition posture for acquiring the reference image as the target acquisition posture.

In 630, a control instruction associated with the image acquisition device may be generated based on the target acquisition protocol and the target acquisition posture.

The control instruction (also referred to as the first control instruction) associated with the image acquisition device is used to cause the image acquisition device to perform a parameter setting based on the target acquisition protocol and the target acquisition posture. The control device may send the control instruction to the image acquisition device 10, and the image acquisition device 10 may adjust the acquisition protocol to the target acquisition protocol and the acquisition posture to the target acquisition posture. In some embodiments, the first control instruction may include the target acquisition protocol and the target acquisition posture. In some embodiments, the first control instruction may include a posture change by adjusting the posture of the image acquisition device to the target acquisition posture.

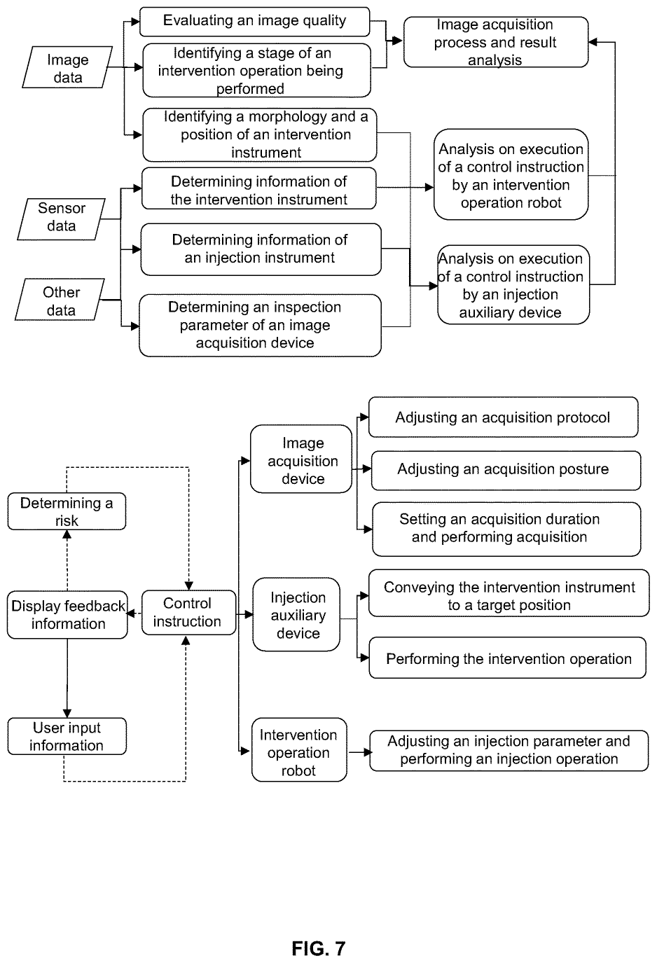

FIG. 7 is a flowchart illustrating an exemplary process of intervention operation control according to some embodiments of the present disclosure.

For example, as shown in FIG. 7, the control device 70 may send image data and an inspection parameter fed back by the image acquisition device 10, and sensor data (e.g., first sensor data and second sensor data) fed back by the intervention operation robot 20 and the injection auxiliary device 30 to the display device 40 for display.

In some embodiments, the feedback information may further include built-in parameters of the image acquisition device 10, the intervention operation robot 20, and the injection auxiliary device 30, such as a size, a model, an operation parameter, and other information.

In some embodiments, the control device 70 may evaluate an image quality based on the image data fed back by the image acquisition device 10, and/or identify a stage of the intervention operation being performed and a morphology and a position of the intervention instrument. For example, the control device 70 may identify the position of the intervention instrument in a blood vessel based on the image data, and determine whether the intervention instrument is in a safety region (e.g., a region in which a distance between the invention instrument and an inner wall of a blood vessel is greater than or equal to a preset distance threshold). As another example, the control device 70 may obtain information such as a current morphology, a current tortuosity, and a position of the intervention instrument in the blood vessel based on the image data. Furthermore, the control device 70 may determine information associated with an image acquisition process (e.g., whether to perform image acquisition, and an acquisition parameter) and perform result analysis (e.g., determine whether to re-acquire the image, and determine the next stage of the current stage) based on an image quality evaluation result and the stage of the intervention operation being performed.

In some embodiments, the control device 70 may determine information (e.g., a force magnitude of the intervention instrument, a force change curve, a force change mode, a force distribution in space, a force change trend within a preset time period, a movement state of the intervention instrument, etc.) of the intervention instrument gripped by the intervention operation robot 20 based on the sensor data (e.g., the first sensor data) fed back by the intervention operation robot 20, and/or determine information (e.g., an injection speed, etc.) of an injection instrument based on the sensor data (e.g., the second sensor data) fed back by the injection auxiliary device 30. Further, the control device 70 may determine execution information of the control instruction by the intervention operation robot 20 based on the information of the intervention instrument, and determine execution information of the control instruction by the injection auxiliary device 30 based on the information of the injection instrument.

In some embodiments, the control device 70 may send an analysis result (e.g., the execution information of the control instruction by the intervention operation robot 20, the execution of the control instruction by the injection auxiliary device 30, the image acquisition process, etc.) to the display device 40 for display.

As shown in FIG. 7, the control device 70 may determine one or more control instructions based on user input information (e.g., a confirmation operation of the user on the feedback information and the first decision data, or the second decision data input by the user) to control at least one of the image acquisition device, the intervention operation robot, or the injection auxiliary device to perform one or more associated operations.

In some embodiments, the control device may determine whether a current operation of the intervention instrument involves a risk based on the image data and the sensor data. In response to determining that current operation of the intervention instrument involves a risk (e.g., the intervention instrument may damage the blood vessel or other organs if the intervention instrument continues to move along a current path, the distance between the intervention instrument and an endangered organ is less than a preset distance threshold, etc.), the control device 70 may adjust a control strategy of the image acquisition device 10 and/or the intervention operation robot 20. For example, the control device 70 may determine a first control instruction including information such as an acquisition duration adjustment, an acquisition frequency adjustment, or the like, and send the first control instruction to the image acquisition device 10, and/or determine a third control instruction containing information such as a convey angle adjustment, a convey distance adjustment, or the like, and send the third control instruction to the intervention operation robot 20.

By adjusting the acquisition duration and acquisition frequency of the image acquisition device 10, the image data information is increased, and whether the risk is involved in the current operation is determined again based on the image data obtained after the acquisition duration adjustment and the acquisition frequency adjustment, so as to improve the determination accuracy of the risk. By the convey angle adjustment and the convey distance adjustment, the risk can be avoided, and the operation safety of the intervention operation can be improved.

In some embodiments, the control instruction may be completed through logical control. For example, a plurality of reference states may be preset and different reference states correspond to different control instructions. If the current state of any one of the image acquisition device 10, the intervention operation robot 20 or the injection auxiliary device 30 satisfies a reference state, state transfer switching may be triggered. The control device 70 may send the control instruction corresponding to the current state to the corresponding device. For example, the plurality of reference states may include a state reflecting a spatial position of the invention instrument, a state reflecting the force information of the invention instrument, a state reflecting the injection speed of the injection instrument, a state reflecting the injection pressure of the injection instrument, etc. The determination or switching of the control instruction may be implemented by an intelligent algorithm, such as deep learning, reinforcement learning, etc.

The control device 70 may control the image acquisition device 10 to adjust an acquisition protocol and an acquisition posture (i.e., the position and the posture of the image acquisition device), set an acquisition duration, and perform an image acquisition process by sending the first control instruction.

The control device 70 may control the intervention operation robot 20 to convey the intervention instrument to a target position to perform the intervention operation by sending the third control instruction.

The control device 70 may control the injection auxiliary device 30 to adjust an injection parameter and perform an injection operation by sending a second control instruction.

For example, when drug injection is required, the intervention operation robot 20 may convey the intervention instrument to the target position, and the injection auxiliary device 30 may perform the injection operation according to a preset injection parameter. During this process, the control device 70 may control the injection auxiliary device 30 to dynamically adjust the injection parameter according to real-time image data (e.g. an image signal intensity of a drug such as a contrast agent, a position of the intervention instrument, etc.) acquired by the image acquisition device 10, the first sensor data (e.g., the force information and the movement information of the intervention instrument) of the intervention operation robot 20, and the second sensor data (e.g., the current injection dose, the current injection speed, and the current injection pressure) of the injection auxiliary device 30. For example, the control device 70 may generate a second control instruction and send the second control instruction to the injection auxiliary device 30. The injection auxiliary device 30 may adjust the injection parameter based on the second control instruction. After the injection at a first position is completed, the control device 70 may generate a third control instruction and send the third control instruction to the intervention operation robot 20. The intervention operation robot 20 may automatically drive the injection instrument to the next injection position (also referred to as a second position) based on the third control instruction. The control device 70 may generate a second control instruction again to control the injection auxiliary device 30 to dynamically adjust the operation parameter of the injection instrument and perform the injection operation at the second position. The above process is repeated until the injection operation in all positions is completed.

It should be noted that the above descriptions of the processes 400, 500 and 600 are only for illustration and description, and do not limit the scope of application of the present disclosure. For those skilled in the art, various modifications and changes can be made to the processes 400, 500, and 600 under the guidance of the present disclosure. However, these modifications and changes are still within the scope of the present disclosure.

FIG. 8 is a schematic structural diagram illustrating an exemplary intervention operation robot according to some embodiments of the present disclosure. FIG. 9 is a schematic structural diagram illustrating an exemplary instrument conveying member according to some embodiments of the present disclosure.

In some embodiments, as shown in FIG. 8, an intervention operation robot (e.g., the intervention operation robot 20) may include an instrument conveying member 201 and a robotic arm 202. The instrument conveying member 201 may be configured to clamp at least one intervention instrument and convey the intervention instrument to a target position of a subject. The robotic arm 202 may be detachably connected with the instrument conveying member 201 and configured to control a movement of the instrument conveying member 201. The movement of the intervention instrument may include translation and/or rotation. The instrument conveying member 201 may include one or more first convey assemblies (not shown in the figure). Each of the one or more first convey assemblies may be configured to clamp one of the at least one intervention instrument and drive the intervention instrument to translate and/or rotate. Conveying refers to driving the intervention instrument to move by means of translation, such as driving the intervention instrument to move back and forth along an axial direction of the one or more first convey assemblies or to move along an axial of the intervention instrument. The target position of the subject refers to an intermediate position or a final position to which the intervention instrument needs to move during the intervention operation. For example, the target position may be a target blood vessel, a lesion site, a region of interest (ROI) of the subject, a position that needs to reach at a certain moment in the process of entering the ROI, etc. More descriptions regarding the intervention instrument may be found in the related descriptions of FIG. 1.

The instrument conveying member 201 may be in various shapes. For example, as shown in FIG. 8 and FIG. 9, the instrument conveying member 201 may be a ship-like structure. The instrument conveying member 201 can be in other shapes that are convenient for movement, such as a strip-like shape, an ellipsoid-like shape, etc.

In some embodiments, when the intervention operation is performed, a catheter may be conveyed to the target position through the instrument conveying member 201 to establish a treatment channel, and then a specific intervention instrument such as a guidewire, a percutaneous balloon or a vascular stent may be introduced into the target position of a human body.

In some embodiments, as shown in FIG. 9, a mounting structure 101 may be provided on the instrument conveying member 201. The robotic arm 202 may be detachably mounted on the mounting structure 101 of the instrument conveying member 201 in various ways, such as detachable mounting by screws. The mounting structure 101 may be located on a side of the instrument conveying member 201, such as at a center region, a position of ⅔, a position of ¾, or the like, of an upper edge of the side.

In some embodiments, as shown in FIG. 8, the robotic arm 202 may be slidably mounted on a medical bed 60. Before the intervention operation begins, the robotic arm 202 may control the instrument conveying member 201 to move to a preset position. The preset position may be determined according to the actual situation of the intervention operation. For example, a position close to the subject or a position close to a lesion of the subject may be determined as the preset position according to a position and/or a posture of the subject on the medical bed 60. After the robotic arm 202 moves to the preset position, the robotic arm 202 remains stationary, and the instrument conveying member 201 may convey the gripped intervention instrument to the target position.

In some embodiments, the robotic arm 202 may drive the instrument conveying member to the preset position by controlling the instrument conveying member 201 to perform at least one of rotation, translation (e.g., upward and downward movements, left and right movements, etc.), etc. In some embodiments, the robotic arm 202 may drive the intervention instrument to the target position by controlling the instrument conveying member 201 to perform at least one of rotation, translation (e.g., upward and downward movements, left and right movements, etc.), etc. For example, after a catheter is clamped in a Y-valve structure of the instrument conveying member 201, the robotic arm 202 may drive the instrument conveying member 201 to move back and forth along an axis of the instrument conveying member 201 or rotate around the axis thereof to convey the clamped catheter to the target position of the subject. The catheter may be used for another catheter (e.g., another catheter with a diameter less than a diameter of the catheter), a guide wire, or other intervention instruments to penetrate through.

In some embodiments, the robotic arm 202 may include at least one joint, and the robotic arm 202 may drive the instrument conveying member 201 to move by rotation of the at least one joint. For example, as shown in FIG. 8, the robotic arm 202 may include a first joint 211, a second joint 212, a third joint 213, a fourth joint 214, and a fifth joint 215 which are connected in sequence, and the fifth joint 215 may be connected with the instrument conveying member 201. The first joint 211 may rotate a first preset angle in a horizontal plane (e.g., a plane where an X-Y axis is located in the figure). The second joint 212 may rotate a second preset angle in the horizontal plane. The third joint 213 may rotate a third preset angle in a vertical plane (e.g., a plane where a Y-Z axis is located in the figure). The fourth joint 214 may rotate a fourth preset angle in the horizontal plane. The fifth joint 215 may rotate a fifth preset angle in the vertical plane (e.g., a plane where an X-Z axis is located). Merely by way of example, the first preset angle may be 360°, 320°, 300°, 270°, 250°, 230°, 200°, 180°, etc. As another example, the second preset angle may be ±180°, ±170°, ±163°, ±150°, ±120°, ±100°, ±90°, etc. As another example, the third preset angle may be in a range of 0°-45°, 0°-47°, 0°-50°, 0°-60°, 0°-70°, 0°-25°, 0°-15°, 0°-30°, 0°-17°, etc. As another example, the fourth preset angle may be ±180°, ±170°, ±163°, ±150°, ±120°, ±100°, ±90°, etc. As another example, the fifth preset angle may be in a range of 0°-45°, 0°-47°, 0°-50°, 0°-60°, 0°-70°, 0°-30°, 0°-27°, 0°-23°, 0°-25°, etc.

In some embodiments, as shown in FIG. 8, the robotic arm 202 may further include a power device 260. The power device 260 may be configured to provide power for the rotation of each joint of the robotic arm 202.

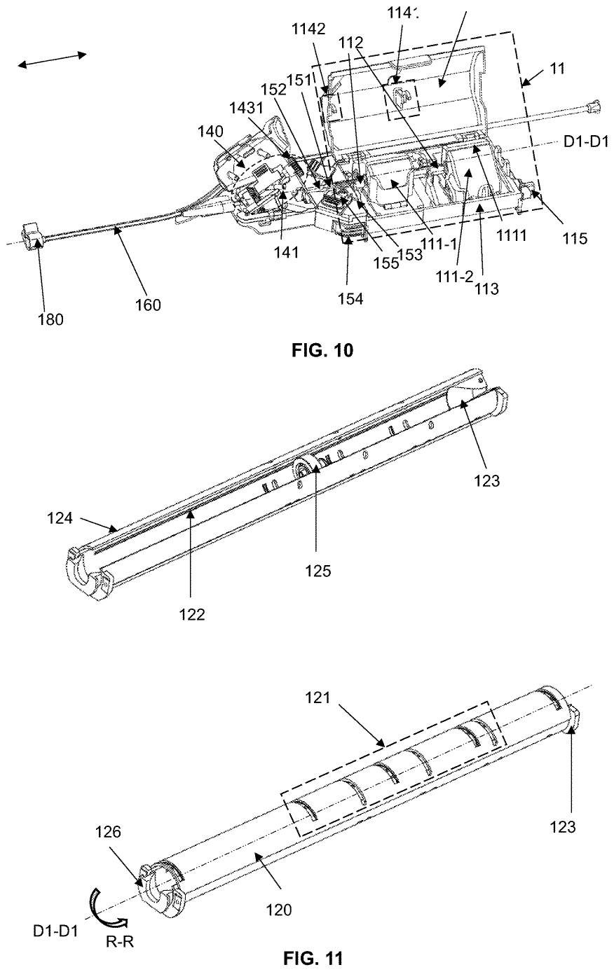

FIG. 10 is a schematic structural diagram illustrating another exemplary instrument conveying member according to some embodiments of the present disclosure.

In some embodiments, one or more first convey assemblies may include at least two clamping members. The at least two clamping members may be configured to clamp an intervention instrument or release clamping the intervention instrument. In response to determining that one of the at least two clamping members clamps the intervention instrument, the other of the at least two clamping members may release clamping the intervention instrument. When one of the at least two clamping members clamps the intervention instrument, the clamping member may be configured to drive the intervention instrument to move or fix the intervention instrument. Taking the one or more first convey assemblies including two clamping members as an example, as shown in FIG. 10, one or more first convey assemblies 11 may include a clamping member 111-1 and a clamping member 111-2. The clamping member 111-1 and the clamping member 111-2 may be configured to clamp the intervention instrument or release clamping the intervention instrument. In response to determining that the clamping member 111-1 clamps the intervention instrument, the clamping member 111-2 releases clamping the intervention instrument; or in response to determining that the clamping member 111-2 clamps the intervention instrument, the clamping member 111-1 releases clamping the intervention instrument.

By providing the two or more clamping members, the operation mode of the two or more clamping members alternately driving the movement of the intervention instrument can realize the continuity of conveying or withdrawing of the intervention instrument. In addition, the operation mode of the two or more clamping members alternately driving the movement of the intervention instrument has no limitation to the conveying or withdrawing distance of the intervention instrument, and the intervention instrument can be conveyed or withdrawn for a long distance without using a long guide rail. The overall device is compact and compact, thereby saving the space of the operating room.

In some embodiments, the instrument conveying member 201 includes two or more first convey assemblies, and each of the two or more first convey assemblies may include two or more clamping members. For example, the instrument conveying member 201 may include two first convey assemblies, and each of the two first convey assemblies may include two clamping members. In some embodiments, when the instrument conveying member 201 includes two or more first convey assemblies, at least one of the two or more first convey assemblies may include only one clamping member. For example, when the instrument conveying member 201 includes two first convey assemblies, each of the two first convey assemblies may include only one clamping member. As another example, the instrument conveying member 201 includes three first convey assemblies, each of two first convey assemblies of the three first convey assemblies may include only one clamping member, and the other of the three first convey assemblies may include two clamping members.

In some embodiments, as shown in FIG. 10, a mounting channel 1111 may be disposed inside the clamping member, and the mounting channel 1111 may be configured to mount the intervention instrument (e.g., a catheter). The mounting channel may be arranged along an axial direction of the instrument conveying member 201.

In some embodiments, the two or more clamping members may be movably arranged along the axial direction of the instrument conveying member 201 (e.g., a direction of a D1-D1 axis in FIG. 10). The movement direction of the clamping member that clamps the intervention instrument may be opposite to a movement direction of the clamping member that releases clamping the intervention instrument (e.g., the clamping member that clamps the intervention instrument and the clamping member that releases clamping the intervention instrument may move in opposite directions along central axes of the one or more first convey assemblies, respectively). The clamping member that clamps the intervention instrument and the clamping member that releases clamping the intervention instrument may move synchronously or asynchronously. That is, the clamping member that clamps the intervention instrument and the clamping member that releases clamping the intervention instrument may move synchronously or asynchronously in opposite directions along the central axes of the one or more first convey assemblies.

In some embodiments, each of the one or more first convey assemblies may include a support substrate, and the one or more clamping members may be movably mounted on the support substrate. As shown in FIG. 10, a support substrate 113 may be disposed at a bottom portion of each of the one or more first convey assemblies 11, and the clamping member 111-1 and the clamping member 111-2 may be movably mounted on the support substrate 113.

The one or more clamping members may move within a preset travel range along the axes of the one or more first convey assemblies. The one or more clamping members may move within the preset travel range, that is, the one or more clamping members may move forward or backward within a certain length range along the axes of the one or more first convey assemblies. For example, the clamping member 111-1 or the clamping member 111-2 may move back and forth within a range of 20 cm along a central axis D1-D1 of the one or more first convey assemblies.

It should be noted that the preset travel range of the two or more clamping members of the one or more first convey assemblies may be flexibly determined according to actual needs, such as 7 cm, 10 cm, 15 cm, 20 cm, 25 cm, 30 cm, etc., which is not limited in the present disclosure. The preset travel ranges corresponding to two adjacent clamping members may be arranged at intervals along the axes of the one or more first convey assemblies without an overlapping portion; or the preset travel ranges corresponding to the two adjacent clamping members may have an overlapping portion. In addition, sizes of the preset travel ranges corresponding to the clamping members may be equal or unequal.

In some embodiments, a channel may be provided inside the instrument conveying member 201 for the entry of the intervention instrument (e.g., a catheter, a guidewire, etc.), such that the at least one intervention instrument may enter the one or more first convey assemblies to be clamped by the at least two clamping members.

In some embodiments, one of the one or more first convey assemblies may include a protective cover 114. The protective cover may be configured to cover the two or more clamping members to prevent dust from entering the one of the one or more first convey assemblies to contaminate the at least one intervention instrument clamped by the clamping members, and prevent an external force from damaging the clamping members or the intervention instrument.

In some embodiments, as shown in FIG. 10, the instrument conveying member 201 may further include a vascular sheath fixing member 180. The vascular sheath fixing member 180 may be configured to fix a vascular sheath inserted into the subject to the instrument conveying member 201.

In some embodiments, one of the one or more first convey assemblies may include an auxiliary clamping structure 115. The auxiliary clamping structure 115 is may be configured to assist a doctor in temporarily clamping and fixing the at least intervention instrument when the catheter, the guidewire, or other intervention instruments are mounted or replaced, so as to prevent the at least one intervention instrument from slipping.

For example, as shown in FIG. 10, each of the one or more first convey assemblies may include a clamping member 111-1 and a clamping member 111-2 which are arranged at intervals along a central axis D1-D1. When a first convey assembly conveys the intervention instrument, the clamping member 111-1 and the clamping member 111-2 may operate alternately. In the process of clamping the intervention instrument by the clamping member 111-1 and moving to the target position along the central axis D1-D1, the clamping member 111-2 may release clamping the intervention instrument and remain stationary. After the clamping member 111-1 moves a first preset distance (less than or equal to the preset travel range of the clamping member 111-1) along the central axis D1-D1, the clamping member 111-1 may release clamping the intervention instrument and return to a first initial position (in this process, the intervention instrument is still in a clamping space of the clamping member 111-2); the clamping member 111-2 may clamp the intervention instrument and move to the target position, at this time the clamping member 111-1 may remain stationary (in this process, the intervention instrument is still in a clamping space of the clamping member 111-1); after the clamping member 111-2 moves a second preset distance (less than or equal to the preset travel range of the clamping member 111-2) along the central axis D1-D1, the clamping member 111-2 may release clamping the intervention instrument and return to a second initial position, and the clamping member 111-1 may clamp the intervention instrument again and move to the target position along the central axis D1-D1. The above process may be repeated until the intervention instrument is moved to the target position of the subject. The first preset distance and the second preset distance may be equal or unequal.

The two or more clamping members of the one or more first convey assemblies may be configured to drive the intervention instrument to withdraw from the target position of the subject. In this case, a movement direction of the one or more first convey assemblies may be opposite to the movement direction of the one or more first convey assemblies for conveying the intervention instrument to the target position, so as to take the intervention instrument out of the target position of the subject. In a stationary state, the two or more clamping members may also clamp the intervention instrument to fix the intervention instrument and prevent the intervention instrument from slipping or moving.

In some embodiments, the instrument conveying member may clamp one of the at least one intervention instrument and drive the intervention instrument to rotate by clamping through the at least two clamping members. For example, as shown in FIG. 10, the clamping member 111-1 or the clamping member 111-2 may clamp the intervention instrument and rotate the clamping member along the D1-D1 axis at a preset angle. The preset angle may be determined according to an actual convey situation of the intervention instrument. For example, the preset angle may be 10 degrees, 20 degrees, 30 degrees, 90 degrees, 180 degrees, 210 degrees, 230 degrees, 270 degrees, etc. The position of the guidewire or the catheter in a vertical plane may be changed by rotating a certain angle. If there is resistance during the entry of the guide wire, factors causing the resistance may be avoided by rotating a certain angle, thereby continuing the forward movement.

FIG. 11 is a schematic structural diagram illustrating an exemplary support sleeve according to some embodiments of the present disclosure.

In some embodiments, as shown in FIG. 9 and FIG. 11, the instrument conveying member 201 may include two or more first convey assemblies, and the two or more first convey assemblies may be connected through at least one support sleeve 120.

The two or more first convey assemblies and the at least one support sleeve 120 may be arranged along an axial direction of the instrument conveying member 201. For example, as shown in FIG. 9, the instrument conveying member 201 may include two first convey assemblies, and the two first convey assemblies may be connected through a support sleeve 120. Two ends of the support sleeve 120 may be fixed to the two first convey assemblies, respectively.

In some embodiments, different first convey assemblies may convey the same or different intervention instruments. For example, when the instrument conveying member includes two first convey assemblies, one of the two first convey assemblies is used to convey the catheter to the target position, the guidewire may be conveyed from the catheter to the target position through the other of the two first convey assemblies, and at this time, each of the two first convey assemblies may include two or more clamping members. Preferably, each of the two first convey assemblies may include two clamping members, which can minimize the volume of the first convey assemblies while achieving consecutive conveying.

The support sleeve 120 may have a hollow structure, and the inner diameter of the support sleeve 120 may be greater than the outer diameter of the intervention instrument. The intervention instrument (e.g., the catheter, the guidewire, etc.) may penetrate through the support sleeve 120. The intervention instrument may be bent inside the support sleeve 120. In some embodiments, a difference between the inner diameter of the support sleeve 120 and the outer diameter of the intervention instrument may be less than a preset threshold, thereby reducing the volume of the support sleeve. For example, the inner diameter of the support sleeve 120 may be slightly greater than an outer diameter of an intervention instrument with the largest diameter of the at least one intervention instrument.

In some embodiments, inner diameters, lengths, etc., of a plurality of support sleeves 120 may be the same or different.

By providing a plurality of first convey assemblies, the types of the at least one intervention instrument being conveyed can be expanded; by increasing the clamping members, clamping contact surfaces of the one or more first convey assemblies to the at least one intervention instrument can be increased, thereby improving the conveying stability. By providing the support sleeve 120, the plurality of first conveying assemblies can be connected, and support and guidance can be provided for the catheter/guidewire, or other intervention instruments between the plurality of first conveying assemblies. The inner diameter of the support sleeve is small, such that the clamped intervention instrument can be effectively wrapped, better support and guidance performance can be realized while minimizing the volume of the support sleeve.

In some embodiments, as shown in FIG. 11, one of the at least one support sleeve 120 may include a plurality of preset apertures 121 distributed along an axial direction of the support sleeve 120. The length of the support sleeve may be adjusted by breaking the support sleeve along one of the preset apertures at different positions.

The preset aperture 121 is a curved hole provided on a surface of one of the at least one support sleeve 120 along the circumference of the support sleeve. Hole lengths of the plurality of preset apertures 121 may be the same or different. A distance between adjacent preset apertures 121 of the plurality of preset apertures 121 along the axial direction of the at least one support sleeve 120 may be the same or different. The hole length refers to a length of the preset aperture along the circumference direction (e.g., the R-R direction in FIG. 11).

In some embodiments, the total length of the at least one support sleeve may be determined based on scenario requirements.

In some embodiments, the length of a support sleeve may be adjusted by other settings. For example, the length of a support sleeve may be adjusted by providing buckles and slots at different positions. As another example, the length of the support sleeve may be adjusted by rotation through a threaded connection.

By providing the plurality of preset apertures 121, the length of a support sleeve may be adjusted, so as to satisfy the support requirements for intervention instruments of different lengths.

In some embodiments, as shown in FIG. 11, one of the at least one support sleeve 120 may include a guide groove 122, a guide component 123, a screw cover 124, and a multi-directional limiting structure 125. The guide component 123 may be configured to determine an extension direction of a target intervention instrument penetrating through the support sleeve. The multi-directional limiting structure 125 may be configured to locate a radial position of the target intervention instrument penetrating through the support sleeve 120 within the support sleeve 120. The guide groove 122 may be configured to assist the multi-directional limiting structure 125 in moving within the support sleeve 120.

The guide groove 122 may be disposed on an inner wall of the support sleeve 120 along an axial direction of the support sleeve 120, and the guide groove 122 and each of the plurality of preset apertures 121 may have no overlapping portion. The guide component 123 may be disposed on at least one end of the support sleeve 120.

By providing the multi-directional limiting structure, the guide groove and the guide component, the target intervention instrument can penetrate through the support sleeve smoothly, and the movement direction of the intervention instrument can be corrected. By providing the multi-directional limiting structure, the guide groove and the guide component, the intervention instrument can be prevented from shaking in the catheter and can be prevented from bending. For example, by fixing the guidewire through the multi-directional limiting structure, the guidewire can be prevented from bending, making it easier to convey the intervention instrument to the target position.

In some embodiments, the screw cover 124 may rotate around a central axis of the support sleeve 120, and a side of the support sleeve 120 may form a closed structure or an open structure through the rotation. The screw cover 124 may further help the target intervention instrument to penetrate through the support sleeve 120 smoothly. In some embodiments, the guide groove 122 may be disposed on an inner side of the other half structure of the support sleeve 120 matching the screw cover 124. That is, the guide groove 122 may be disposed on an inner side of a relatively fixed main structure of the support sleeve 120.

In some embodiments, the multi-directional limiting structure 125 may be disposed at a middle region inside the support sleeve 120. The multi-directional limiting structure 125 has multiple degrees of freedom, so as to adjust the movement direction of the intervention instrument. For example, the multi-directional limiting structure 125 may include a first degree of freedom for rotation around an axis D1-D1 and a second degree of freedom for translation along the axis D1-D1. The multi-directional limit structure 125 may be connected to a tail end of the catheter. For example, when the catheter and the guidewire are conveyed simultaneously, a first convey assembly of the one or more first convey assemblies located at a near end may clamp the catheter, the tail end of the catheter may be connected with the multi-directional limiting structure 125, and multi-directional limiting structure 125 may move back and forth or rotate along with the movement of the catheter. A first convey assembly of the one or more first convey assemblies located at a far end may convey the guidewire. The guide groove 122 may match the multi-directional limiting structure 125 to assist a central axis of one of the at least two clamping members to maintain flush with an axis of a first channel during the movement.

In the embodiment, one of the at least one support sleeve 120 may further include a fixing structure 126. The fixing structure 126 may be disposed at two ends of the support sleeve 120. The fixing structure 126 may be configured to fix the position of the support sleeve 120. For example, the support sleeve 120 may be fixedly connected with one of the one or more first convey assemblies or another support sleeve through the fixing structure 126.

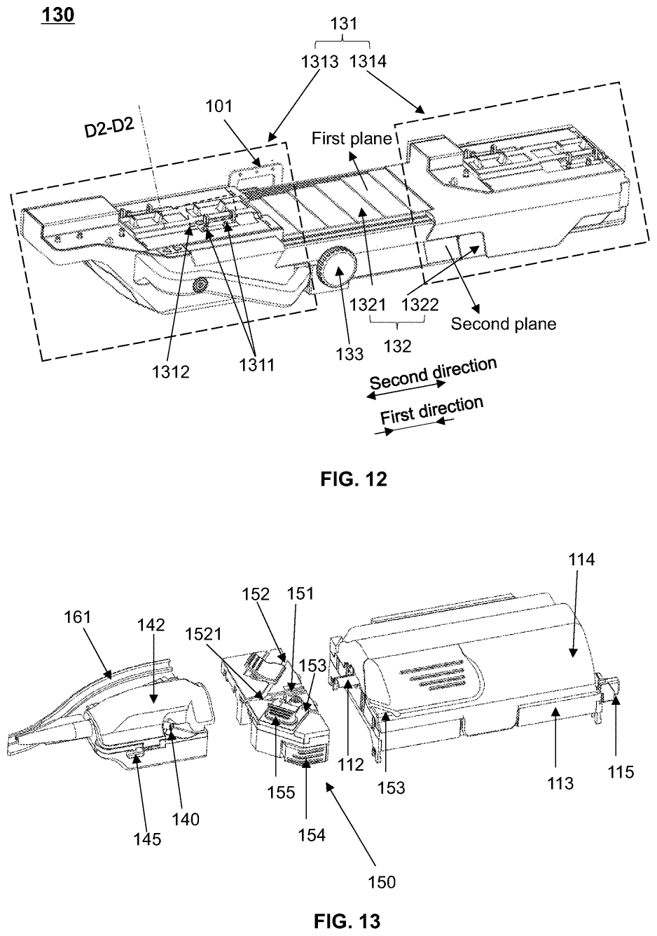

FIG. 12 is a schematic structural diagram illustrating an exemplary retractable base according to some embodiments of the present disclosure.

In some embodiments, as shown in FIG. 12, an instrument conveying member may further includes a retractable base 130 detachably connected with one or more first convey assemblies and configured to assist the one or more first convey assemblies in conveying at least one intervention instrument.

The retractable base 130 is a retractable base of the instrument conveying member. The retractable base 130 is retractable along an axial direction (e.g., a D2-D2 direction in FIG. 12) of the instrument conveying member. The retractable base 130 may be a ship-like shape or other shapes. In some embodiments, the D2-D2 direction may be perpendicular to a first plane, and the one or more first convey assemblies may be mounted on the retractable base 130 along the D2-D2 direction. More descriptions regarding the first plane may be found in the related descriptions of FIG. 12 below.

The retractable base 130 may be detachably connected with the one or more first convey assemblies in various ways, such as by screws or buckles.

In some embodiments, the retractable base 130 may be in transmission connection with the one or more first convey assemblies. For example, an upper surface of the retractable base 130 may be provided with a protruding shaft, a transmission shaft may be provided below the one or more first convey assemblies, and the protruding shaft and the transmission shaft may be meshed/gearing with each other. A drive unit may be disposed inside the retractable base 130. The drive unit may be configured to transmit power to the protruding shaft on the upper surface of the retractable base 130 to transmit the power to the one or more first convey assemblies through the transmission shaft meshed with the protruding shaft, such that one of the at least one intervention instrument may be clamped and driven to rotate by clamping through the clamping member 111-1 or the clamping member 111-2. The protruding shaft and the transmission shaft may be configured as internal and external hexagonal structures nested with each other, internal and external gear structures meshed with each other, etc. Each of the clamping members may include at least two transmission shafts and a connection direction between the at least two transmission shafts is parallel to the axis D1-D1. The transmission shafts in each of the one or more first convey assemblies may be evenly distributed/arranged on two sides of the axis D1-D1. The protruding shaft may perform reciprocating translation movement along the axis D1-D1. The movement stroke of the protruding shaft may be equal to or greater than a travel range of each of the clamping members.

A protruding shaft on the upper surface of the retractable base 130 may be provided with a measurement module or several measurement modules. The count of the measurement modules may match the count of the protruding shafts. The connection direction of the measurement module and the movement stroke of the measurement module in the D1-D1 direction may match those of the protruding shaft. The measurement module may be configured to control a clamping force and a rotation accuracy of one of the clamping members, and feedback and monitor a real-time clamping force of the one of the clamping members during operation.

In some embodiments, a bottom surface of the support substrate 113 of each of the one or more first convey assemblies may be contacted and bonded to the upper surface of the retractable base 130 and can realize mechanical locking to ensure the stability of the connection.

The retractable base may assist in conveying the at least one intervention instrument, thereby further increasing the convey range. Meanwhile, the design of the retractable base can reduce and simplify the overall structure of the intervention operation robot.

In some embodiments, as shown in FIG. 12, the retractable base 130 may include a body 131 configured to assist the one or more first convey assemblies in conveying the at least one intervention instrument. The body 131 may include at least two portions. The at least the two portions may be connected through at least one retractable assembly. Takes the body 131 including two portions as an example. As shown in FIG. 12, the body 131 may include a first body portion 1313 and a second body portion 1314, and the first body portion 1313 and the second body portion 1314 may be connected through a retractable assembly. The retractable assembly may include a retractable substrate 132 and a control member 133. The control member 133 may be in a transmission connection with the retractable substrate 132. Two ends of the retractable substrate 132 may be respectively connected with the first body portion 1313 and the second body portion 1314.

The body 131 is a functional component on the retractable base for supporting the first convey assembly.

The retractable assembly is a structural assembly that completes retraction and expansion. The retractable substrate 132 refers to a retractable part of the retractable assembly. The control member 133 refers to a part of the retractable assembly that controls the retractable substrate 132 to perform retraction and expansion.

The control member 133 and the retractable substrate 132 may be in transmission connection in various ways, such as a transmission connection through a gear, a chain, or a coupler.

In some embodiments, as shown in FIG. 12, the retractable substrate 132 may include a first moving member 1321 formed by connecting a plurality of first plates and a second moving member 1322 formed by connecting a plurality of second plates. As shown in FIG. 12, the first moving member 1321 may be located on a top of the retractable base 130, the second moving member 1322 may be located one a side of the retractable base 130, and two ends of the first moving member 1321 and the second moving member 1322 may be respectively connected with the first body portion 1313 and the second body portion 1314. When the retractable substrate moves in a first direction, the plurality of first plates may be stacked layer by layer on a first plane, and the plurality of second plates may be stacked layer by layer on a second plane; and when the retractable substrate moves in a second direction, the plurality of stacked first plates and the plurality of stacked second plates may be spread out. The retractable substrate 132 shown in FIG. 12 is in an extended state.

The first plate and the second plate may be a rectangular shape or other regular shapes. The plurality of first plates may slide relative to each other along an axial direction of the retractable base 130, and a sliding distance of the first plates may be less than the length of each of the first plates in the axial direction of the retractable base 130. The plurality of second plates may slide relative to each other along the axial direction of the retractable base 130, and the sliding distance of the second plates may be less than the length of each of the second plates in the axial direction of the retractable base 130. The sizes of the first plates and the second plates may be the same or different. In some embodiments, the second moving member formed by connecting the plurality of second plates layer by layer may be provided on two sides of the retractable substrate 132.

The first direction refers to a direction in which the at least two portions of the body are close to each other. The second direction refers to a direction in which the at least two portions of the body are away from each other.

The first plane refers to a top plane of the retractable base 130 where the first moving member 1321 is located. The second plane refers to a side surface of the retractable base 130 where the second moving member 1322 is located.

The retraction and expansion of the first moving member 1321 and the second moving member 1322 by adjusting the control member 133 can drive the at least two portions of the body to be close to or away from each other, thereby assisting in the position adjustment of the intervention instrument.

In some embodiments, the retractable substrate may include a rolling gate and a motor. For example, the motor may be located in the first body portion and fixedly connected with the first body portion. One end of the rolling gate may be fixed on the motor, and the other end of the rolling gate may be connected with the second body portion. The rolling gate may include a straightened state and a retracted state. When the motor rotates along the first direction, the rolling gate may be in the retracted state, and when the motor moves along the second direction, the rolling gate may be in the straightened state.

In some embodiments, as shown in FIG. 12, each of the at least two portions of the body 131 may include: at least one measurement component 1312 configured to measure a translation distance and/or a rotation angle of the first convey assemblies connected with the body 131; and at least one power structure 1311 configured to drive the first convey assemblies connected with the body to move.

The measurement component 1312 and the power structure 1311 may correspond to the clamping members, respectively. For example, when the each of the one or more first convey assemblies includes two clamping members, the first convey assembly may include two measurement components 1312 for measuring a rotation angle and/or a movement distance of the two clamping members, respectively. Meanwhile, when the each of the one or more first convey assemblies includes two clamping members, the first convey assembly may include two power structures 1311 for driving the two clamping members to move.

In some embodiments, the measurement component 1312 may include an angle sensor and/or a displacement sensor, etc. The power structure 1311 may include a drive motor, etc.

In some embodiments, the one or more first convey assemblies may be connected with the body 131 through the one or more protruding shafts and the one or more transmission shafts which are in transmission connection through meshing, which may be found in the related descriptions above.

In some embodiments, as shown in FIG. 9, the power structure 1311 may drive the one or more first convey assemblies to rotate 0-360 degrees around a D3-D3 axis.

In some embodiments, the power structure 1311 may drive the clamping members of the one or more first convey assemblies to rotate or move, to drive the at least one intervention instrument to rotate or move simultaneously.

In some embodiments, as shown in FIG. 12, the at least two portions of the body 131 may include a first body portion 1313 and a second body portion 1314. The first body portion 1313 and the second body portion 1314 may be connected through a retractable assembly. The control member 133 may be configured to drive the retractable substrate 132 to move in a first direction or a second direction. In the first direction, a distance between the first body portion 1313 and the second body portion 1314 may be reduced. In the second direction, the distance between the first body portion 1313 and the second body portion 1314 may be increased.

When the control member 133 is configured to drive the retractable substrate 132 to move in the first direction, a plurality of first plates may be stacked layer by layer on a first plane, a plurality of second plates may be stacked layer by layer on a second plane, the retractable substrate 132 may retract, and the distance between the first body portion 1313 and the second body portion 1314 may be reduced. When the control member 133 is configured to drive the retractable substrate 132 to move in the second direction, the stacked first plates and the stacked second plates may spread out, the retractable substrate 132 may extend, and the distance between the first body portion 1313 and the second body portion 1314 may be increased.

In some embodiments, as shown in FIG. 10, the instrument conveying member may include a Y-valve assembly 140. In response to determining that drug injection is achieved through the instrument conveying member, during a drug injection process, the Y-valve assembly 140 may be controlled to lock; and after the drug injection is completed, the Y-valve assembly 140 may be controlled to loosen.

The Y-valve assembly 140 is an assembly for supporting the intervention instrument. In some embodiments, as shown in FIG. 9 and FIG. 10, the Y-valve assembly 140 may include a Y-valve 141, a Y-valve protective cover 142, a first power structure 143, and a second power structure 144. The Y-valve assembly 140 may include a first inlet, a second inlet, and an outlet. One end of the first inlet and one end of the second inlet may intersect and communicate with the outlet such that the at least one intervention instrument and/or drug may enter the target position of the subject. The second inlet may be used for drug injection, which is also referred to as an injection inlet.

The Y-valve 141 is a valve for controlling the locking or loosening of the Y-valve assembly 140. For example, the Y-valve may be a rotary valve. The injection inlet may be configured to connect an injection pipeline. The first inlet is an entrance for the at least one intervention instrument (e.g., the catheter) to enter the Y-valve, which is also referred to as an instrument inlet. The instrument inlet may accommodate a plurality of intervention instruments. The plurality of intervention instruments may enter the Y-valve from the instrument inlet, penetrate through the Y-valve and exit from the outlet. The plurality of intervention instruments may be nested together or placed side by side.

The Y-valve protective cover 142 may be configured to shield and protect an internal structure of the Y-valve assembly 140. The first power structure 143 may be configured to drive the Y-valve assembly 140 to open or lock. For example, the first power structure 143 may drive the Y-valve 141 to rotate to lock or screw the instrument inlet 1431. The second power structure 144 may be configured to drive the intervention instrument connected with the Y-valve to rotate. For example, a tail end of the Y-valve structure may be connected with the catheter and may rotate independently, while the body of the Y-valve structure may remain stationary. The second power structure 144 may be configured to drive the tail end of the Y-valve structure to rotate, thereby driving the catheter to rotate to convey the catheter to the target position of the subject.

In some embodiments, as shown in FIG. 9, the Y-valve assembly 140 may include a second locking member 145. The second locking member 145 may be configured to lock when the Y-valve protective cover 142 is closed and open after the Y-valve protective cover 142 is locked.

FIG. 13 is a schematic diagram illustrating an exploded structure of an instrument conveying member 201 according to some embodiments of the present disclosure.

In some embodiments, the instrument conveying member 201 may further include a second convey assembly 150 located at a proximal end the instrument conveying member 201. The second convey assembly 150 may include a first channel 151, a second channel 152, and a third channel 153. The first channel 151 may be configured to allow a first intervention instrument to penetrate through. The second channel 152 may be configured to allow a second intervention instrument to penetrate through. The third channel 153 may be configured to allow a third intervention instrument to penetrate through.

Each of the first intervention instrument, the second intervention instrument, and the third intervention instrument may include at least one of a catheter, a guidewire, a fast-crossing catheter, or the like.

The first channel 151 may be located in the middle region of the second convey assembly 150 and arranged along an axial direction of the second convey assembly 150. The second channel 152 and the third channel 153 may be located on two sides of the first channel 151, respectively.

The first channel 151, the second channel 152, and the third channel 153 may merge into one channel 1521 at an end portion of a side of the second convey assembly 150 close to a target channel. In some embodiments, the first channel 151, the second channel 152, and the third channel 153 may merge to be connected with the instrument inlet of the Y-valve assembly.

Taking the structure shown in FIG. 13 as an example, the first channel 151 may be located in the middle region of the second convey assembly 150, and the first intervention instrument may be a guidewire. The first channel 151 may allow the guidewire to penetrate through, such that the instrument conveying member may convey the guidewire to a target position of a patient body. The second channel 152 and the third channel 153 may be respectively located on two sides of the first channel 151. The setting of the second channel 152 and the third channel 153 causes the instrument conveying member to convey the guidewire into the patient body while carrying the second intervention instrument in the second channel 152 and the third intervention instrument in the third channel 153 into the patient body simultaneously, so as to realize a convey operation of a plurality of intervention instruments. That is, a synchronous movement of the plurality of intervention instruments is realized.

For example, in a cardiac intervention operation, the second intervention instrument may be a percutaneous balloon, a balloon-expandable stent, and other components. First, the guidewire may be introduced to the target position of the patient body. At this time, the second intervention instrument in the second channel 152 and the third intervention instrument in the third channel 153 may enter the patient body together. The instrument conveying member may stop at a current position, and the balloon-expandable stent may be pushed through the first convey assembly to drive the percutaneous balloon to a release position (i.e., the target position). During this process, the third channel 153 is not used. In a vascular intervention operation, the third channel 153 may be provided with an intervention instrument for preventing intraoperative complications in advance, but the intervention instrument in the third channel 153 does not move relative to the instrument conveying member and the intervention instrument in the first channel 151. For example, in a neural intervention operation, a brain protection device may be placed in the third channel 153 in advance to prevent distal embolism caused by plaque or thrombus detachment.

In some embodiments, a push main board 110 of the second convey assembly may be provided with one or more buckles protruding outward for modular splicing with other modules (e.g., the one or more first convey assemblies) of the instrument conveying member. The shape of each of the one or more buckles may be a trapezoid, a semicircle, or the like, which is not limited in the present disclosure. Alternatively, the one or more buckles may be one or more slots.

In some embodiments, if there are a plurality of catheters and guidewires to be conveyed, and diameters of the catheters and the guidewires are not the same, a first catheter of the catheters with the largest diameter may be conveyed through the Y-valve assembly. The first catheter may be fixed in the Y-valve assembly and conveyed into the target position of the subject through the Y-valve assembly. For example, the whole instrument conveying member 201 may be moved by means of the platform guide rail through the robotic arm, thereby driving the first catheter in the Y-valve assembly to translate, and the second power structure 144 at the tail end of the Y-valve structure may drive the first catheter to rotate, thereby achieving conveying of the first catheter. The first channel 151 may convey a second catheter with a diameter less than the diameter of the first catheter, and a first guidewire. If the first guidewire is stuck in the subject and cannot be moved, the first guidewire may be removed from the first convey assembly and moved to the second channel 152. Meanwhile, a second guidewire may be placed in the first convey assembly and conveyed through the first channel 151. In this case, the first guidewire and the second guidewire may be placed side by side at the junction of the first channel and the second channel. When a fast-crossing catheter needs to be conveyed, the second guidewire may be removed from the first convey assembly to penetrate through a circumferentially closed end of the fast-crossing catheter. The circumferentially unenclosed end of the fast-crossing catheter may be conveyed through the third channel 153. In this embodiment, the second catheter, the second guidewire, and the fast-crossing catheter may be nested in the channel.

In some embodiments, as shown in FIG. 10 and FIG. 13, a target first convey assembly of the one or more first convey assemblies may include a first limiting member 112. The second convey assembly may include a first locking member 154. The target first convey assembly may be detachably connected with the second convey assembly 150.

The target first convey assembly refers to a first convey assembly connected with the second convey assembly.

The first limiting member 112 may be configured to wrap and limit the corresponding first channel after the target first convey assembly is connected with the second convey assembly. The first limiting member 112 may be arranged along the central axis of the first channel 151 and limit a radial displacement of the first channel 151. In some embodiments, as shown in FIG. 10, a first matching member 1141 and a second matching member 1142 may be disposed on an inner side of the protective cover 114. The first matching member 1141 and the second matching member 1142 may match the first limiting member 112. For example, the first matching member 1141 and the second matching member 1142 may be a buckle, respectively. In some embodiments, the protective cover 114 may be a movable cover resembling a semicircle. When the protective cover 114 is in an open state, an opening of the first limiting member 112 may be upward, and the first channel 151 may not be covered by limiting. When the protective cover 114 is in a closed state, the first limiting member 112 may cooperate with the first matching member 1141 and the second matching member 1142 to wrap and limit the first channel 151 at the position of the first limiting member 112, thereby limiting the radial displacement of the intervention instrument clamped by the first convey assembly during the conveying process.

The first locking member 154 may be configured to lock or unlock connection of the retractable base 130 and one or more assemblies disposed thereon (e.g., the one or more first convey assemblies, the second convey assembly, etc.), such that the detachable connection between the one or more assemblies and the retractable base can be achieved. For example, when the second convey assembly is disposed on the retractable base, the retractable base 130 and the second convey assembly may be locked by the first locking member 154; the second convey assembly and the retractable base 130 may be unlocked by moving the first locking member 154 so as to remove the second convey assembly from the retractable base 130.

In some embodiments, the second convey assembly 150 may include a first power member 155. The first power member may be located beside the third channel 153. The retractable base may include a second power member 134. The second convey assembly 150 may be detachably connected with the retractable base 130. In response to determining that the second convey assembly 150 is connected with the retractable base 130, the first power member 155 may be driven to drive a third intervention instrument to move in the third channel 153 by controlling the second power member 134 to move.

The first power member 155 may be configured to provide power for the third target intervention instrument to move in the third channel. The second power member 134 may be configured to drive the first power member 155 to move. The first power member 155 may be in a transmission connection with the second power member 134. In some embodiments, the first power member 155 may include a driving wheel and a driven wheel. The driving wheel and the driven wheel may be respectively arranged on two sides of the third channel. The second power member 134 may be connected with the driving wheel of the first power member 155. When the intervention instrument is conveyed, the driving wheel connected with the second power member 134 may be driven to rotate by controlling the second power member 134 to move. The driving wheel may drive the driven wheel to rotate synchronously, so as to drive the third target intervention instrument in the third channel between the driven wheel and the driving wheel to move along the third channel. In a standby state, the driven wheel and the driving wheel may be separated from each other.

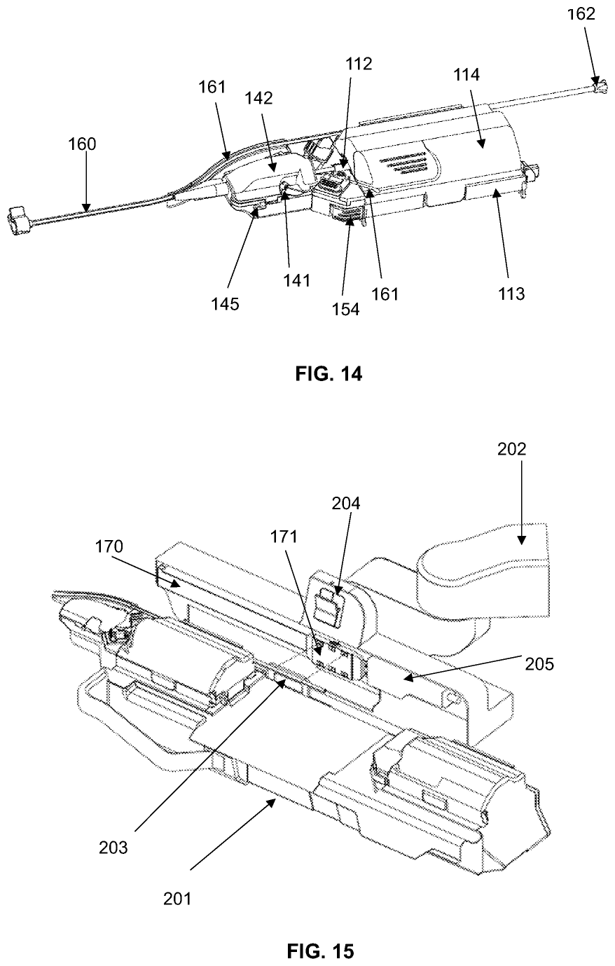

FIG. 14 is a schematic structural diagram illustrating an instrument conveying member according to some embodiments of the present disclosure.

In some embodiments, as shown in FIG. 14, the instrument conveying member 201 may further include a protective sleeve 160 located at a front end of the instrument conveying member 201. The protective sleeve 160 may be made of a flexible material and configured to protect an intervention instrument being conveyed from bending during the conveying process, thereby preventing affecting the conveying effect and efficiency of the intervention instrument.

The protective sleeve 160 may be configured to protect the intervention instrument being conveyed and prevent the intervention instrument from being damaged by friction between the intervention instrument and the front end of the instrument conveying member 201. In some embodiments, the protective sleeve 160 may include a protective sleeve channel 161 and a second limiting member 162. The protective sleeve channel 161 is a mounting channel for the protective sleeve 160 and configured to accommodate and protect the intervention instrument. The second limiting member 162 is a component for limiting the position of the protective sleeve 160. The second limiting member 162 may be fixed on the robotic arm 202 such that the protective sleeve 160 and the robotic arm 202 may remain relatively stationary during the movement of the instrument conveying member 201.

The flexible material may include rubber, silicone or other materials. The protective sleeve can prevent the intervention instrument from bending during the conveying process, thereby preventing affecting the conveying effect and efficiency of the intervention instrument.

FIG. 15 is a schematic diagram illustrating an exploded structure of a platform guide rail according to some embodiments of the present disclosure.

In some embodiments, as shown in FIG. 8 and FIG. 15, the instrument conveying member 201 may further include a platform guide rail 170 configured to support a retractable base. The robotic arm 202 is capable of controlling the instrument conveying member 201 connected thereto to move along the platform guide rail 170.

The platform guide rail 170 may be arranged along an axial direction of the instrument conveying member 201. The platform guide rail 170 may be configured as a slide rail, and the instrument conveying member 201 may slide along the slide rail.

In some embodiments, as shown in FIG. 15, the instrument conveying member 201 may be provided with a guide rail slider 171 matching the platform guide rail, and a first fixing member 203. The first fixing member 203 may be configured to fix the instrument conveying member 201 and the guide rail slider 171. A guide rail drive device 205 may be provided on the instrument conveying member 201. The guide rail drive device 205 may be configured to drive the guide rail slider 171 to move on the platform guide rail 170, so as to drive the instrument conveying member 201 to move. The guide rail drive device 205 may be a motor, etc.

In some embodiments, as shown in FIG. 15, a second fixing member 204 may be provided on the robotic arm 202. The second fixing member 204 may be configured to fix the second limit member 162 to the robotic arm.

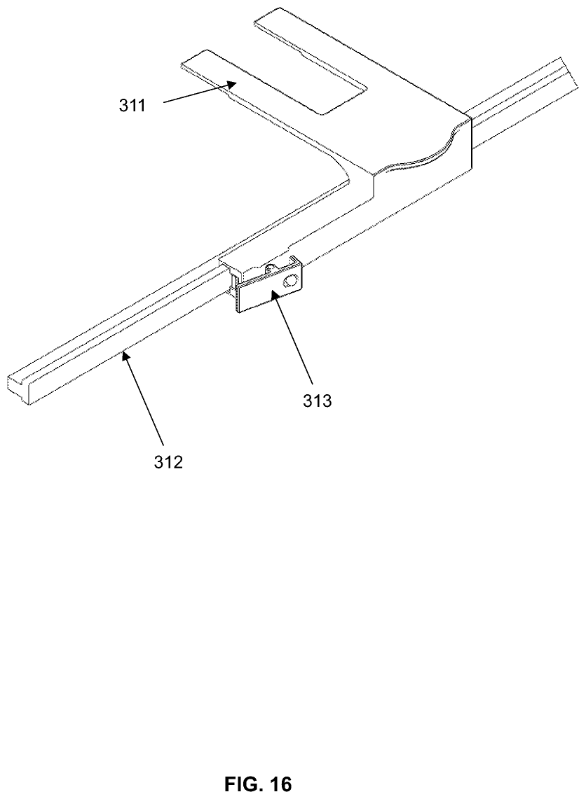

FIG. 16 a schematic diagram illustrating an exploded structure of a support platform according to some embodiments of the present disclosure.

In some embodiments, as shown in FIG. 8 and FIG. 16, an intervention operation robot may further include a support platform 300. The support platform 300 may include a first support assembly 311 detachably connected with the medical bed 60; and a second support assembly 312 slidably connected with the first support assembly. The robotic arm 202 may be connected to the support platform 300 through the second support assembly 312.

The support platform 300 may be configured to connect the medical bed 60 and the intervention operation robot. As shown in FIG. 8, the first support assembly 311 may be detachably connected to an edge of the medical bed 60. The robotic arm 202 may slide on the first support assembly 311 through the second support assembly 312, to adjust the position of the robotic arm.

In some embodiments, as shown in FIG. 16, the support platform 300 may further include a third locking member 313. The third locking member 313 may be configured to lock the robotic arm and the second support assembly 312 to strengthen the connection.

Some embodiments of the present disclosure further provide an intervention operation robot. The intervention operation robot may comprise an instrument conveying member (e.g., the instrument conveying member 201) configured to clamp at least one intervention instrument and convey the intervention instrument to a target position of a subject; and a robotic arm (e.g., the robotic arm 202) detachably connected with the instrument conveying member and configured to control a movement of the instrument conveying member. The instrument conveying member may include one or more first convey assemblies each of which may be configured to clamp one of the at least one intervention instrument and drive the intervention instrument to translate and/or rotate.

In some embodiments, the intervention operation robot may further include a support platform (e.g., the support platform 300). More descriptions regarding the instrument conveying member, the robotic arm, and the support platform may be found in the related descriptions of FIGS. 8-16.

Some embodiments of the present disclosure further provide an intervention instrument convey device configured to clamp an intervention instrument and convey the intervention instrument to a target position of a subject. The intervention instrument convey device may comprise one or more first convey assemblies. Each of the one or more first convey assemblies may be configured to clamp one of at least one intervention instrument and drive the intervention instrument to translate and/or rotate. In some embodiments, the intervention instrument convey device may further comprise a support sleeve (e.g., the support sleeve 120), a retractable base (e.g., the retractable base 130), a Y-valve assembly (e.g., the Y-valve assembly 140), a second convey assembly (e.g., the second convey assembly 150), a protective sleeve (e.g., the protective sleeve 160), a platform guide rail (e.g., the platform guide rail 170), etc. More descriptions may be found in FIGS. 8-16.

Having thus described the basic concepts, it may be rather apparent to those skilled in the art after reading this detailed disclosure that the foregoing detailed disclosure is intended to be presented by way of example only and is not limiting. Various alterations, improvements, and modifications may occur and are intended to those skilled in the art, though not expressly stated herein. These alterations, improvements, and modifications are intended to be suggested by this disclosure and are within the spirit and scope of the exemplary embodiments of this disclosure.

1. A system for intervention operation control, comprising:
an injection auxiliary device configured to assist an injection instrument for injection;
an intervention operation robot configured to convey an intervention instrument;
an image acquisition device configured to acquire one or more images; and
a control device configured to generate one or more control instructions based on feedback information from at least one of the injection auxiliary device, the intervention operation robot, or the image acquisition device, and send the one or more control instructions to at least one of the injection auxiliary device, the intervention operation robot, or the image acquisition device, wherein the one or more control instructions are instruction information for controlling an operation of the injection auxiliary device, the intervention operation robot, and/or the image acquisition device.

2. The system of claim 1, wherein the intervention operation includes a plurality of stages, and the generating one or more control instructions based on feedback information from at least one of the injection auxiliary device, the intervention operation robot and the image acquisition device comprises:
determining, based on the feedback information, a stage of the intervention operation being performed; and
determining the one or more control instructions that match a next stage of the stage of the intervention operation being performed;
wherein the feedback information includes an image acquired by the image acquisition device during the intervention operation, the determining, based on the feedback information, a stage of the intervention operation being performed comprises:
determining a feature analysis result by analyzing the image acquired by the image acquisition device during the intervention operation; and
determining, based on the feature analysis result, the stage of the intervention operation being performed.

3-4. (canceled)

5. The system of claim 1, wherein the feedback information includes an image acquired by the image acquisition device during the intervention operation, the generating one or more control instructions based on feedback information from at least one of the injection auxiliary device, the intervention operation robot, or the image acquisition device comprises:
determining first state information of the intervention operation robot based on the feedback information;
determining, based on the first state information and the image, second state information of the intervention instrument being gripped by the intervention operation robot;
determining, based on the first state information and the second state information, first decision data matching a next stage of a stage of the intervention operation being performed; and
generating the one or more control instructions based on the first decision data.

6. The system of claim 1, wherein the generating one or more control instructions based on feedback information from at least one of the injection auxiliary device, the intervention operation robot, or the image acquisition device comprises:
comparing an image acquired by the image acquisition device with a reference image;
generating, based on a comparison result, a target acquisition protocol and a target acquisition posture of the image acquisition device; and
generating a control instruction associated with the image acquisition device based on the target acquisition protocol and the target acquisition posture, the control instruction being used to cause the image acquisition device to perform a parameter setting based on the target acquisition protocol and the target acquisition posture.

7. The system of claim 1, wherein
during an execution of a corresponding control instruction by at least one of the injection auxiliary device, the intervention operation robot, and the image acquisition device,
the control device is configured to control a target device associated with second decision data to stop executing a control instruction if the second decision data input by a user is received, the target device being at least one of the injection auxiliary device, the intervention operation robot, or the image acquisition device; and
generate a decision instruction based on the second decision data and send the decision instruction to the device associated with the second decision data.

8-24. (canceled)

25. An intervention operation robot, comprising:
an instrument conveying member configured to clamp at least one intervention instrument and convey the at least one intervention instrument to a target position of a subject; and
a robotic arm detachably connected with the instrument conveying member and configured to control a movement of the instrument conveying member;
wherein the instrument conveying member includes one or more first convey assemblies, and each of the one or more first convey assemblies is configured to clamp one of the at least one intervention instrument and drive the intervention instrument to translate and/or rotate.

26. The intervention operation robot of claim 25, wherein each of the one or more first convey assemblies is configured to clamp one of the at least one intervention instrument and drive the intervention instrument to move along an axial direction of the first convey assembly and/or drive the intervention instrument to rotate around an axis of the first convey assembly.

27. The intervention operation robot of claim 25, wherein each of the one or more first convey assemblies includes at least two clamping members, the at least two clamping members are configured to clamp the intervention instrument or release clamping the intervention instrument, and in response to determining that one of the at least two clamping members clamps the intervention instrument, the other of the at least two clamping members releases clamping the intervention instrument.

28. The intervention operation robot of claim 25, wherein a count of the one or more first convey assemblies of the instrument conveying member exceeds 1, the first convey assemblies are connected through at least one support sleeve, and the first convey assemblies are configured to convey different types of target intervention instruments;
the one of the at least one support sleeve includes a plurality of preset apertures distributed along an axial direction of the support sleeve, and a length of the support sleeve is adjusted by breaking the support sleeve along one of the plurality of preset apertures at different positions.

29. (canceled)

30. The intervention operation robot of claim 28, wherein one of the at least one support sleeve includes a multi-directional limiting structure, a guide groove and a guide component, the guide component is configured to determine an extension direction of a target intervention instrument penetrating through the support sleeve, the multi-directional limiting structure is configured to locate a radial position of the target intervention instrument penetrating through the support sleeve within the support sleeve, and the guide groove is configured to assist the multi-directional limiting structure in moving within the support sleeve.

31. The intervention operation robot of claim 25, wherein the instrument conveying member includes:
a retractable base, detachably connected with the one or more first convey assemblies and configured to assist the one or more first convey assemblies in conveying the at least one intervention instrument;
the retractable substrate includes a body configured to assist the one or more first convey assemblies in conveying the at least one intervention instrument, the body including at least two portions, the at least two portions being connected through at least one retractable assembly,
wherein each of the at least one retractable assembly includes a retractable substrate and a control member, the control member is in a transmission connection with the retractable substrate, and two ends of the retractable substrate are respectively connected with the at least two portions of the body.

32. (canceled)

33. The intervention operation robot of claim 31, wherein the retractable substrate includes a first moving member formed by connecting a plurality of first plates and a second moving member formed by connecting a plurality of second plates, the first moving member is located on a top of the retractable base, the second moving member is located on a side of the retractable base, and two ends of the first moving member and the second moving member are respectively connected with the at least two portions of the body;
when the retractable substrate moves in the first direction, the plurality of first plates are stacked layer by layer on a first plane, and the plurality of second plates are stacked layer by layer on a second plane; and
when the retractable substrate moves in the second direction, the plurality of stacked first plates and the plurality of stacked second plates are spread out.

34. The intervention operation robot of claim 31, wherein each of the at least two portions of the body includes:
at least one measurement component configured to measure a translation distance and/or a rotation angle of the first convey assemblies connected with the body; and
at least one power structure configured to drive the first convey assemblies connected with the body to move.

35. The intervention operation robot of claim 25, wherein the at least two portions of the body include a first body portion and a second body portion, the first body portion and the second body portion are connected through the retractable assembly, the control member is configured to drive the retractable substrate to move in the first direction or the second direction, and in the first direction, a distance between the first body portion and the second body portion is reduced, and in the second direction, the distance between the first body portion and the second body portion is increased.

36. The intervention operation robot of claim 25, wherein the instrument conveying member further includes a Y-valve assembly, and instrument conveying member, during a drug injection process, the Y-valve assembly is controlled to lock; and
after the drug injection is completed, the Y-valve assembly is controlled to loosen.

37. The intervention operation robot of claim 25, wherein the instrument conveying member further includes a second convey assembly located at a proximal end of the instrument conveying member, the second convey assembly includes a first channel, a second channel, and a third channel, the first channel is configured to allow a first intervention instrument to penetrate through, the second channel is configured to allow a second intervention instrument to penetrate through, and the third channel is configured to allow a third intervention instrument to penetrate through.

38. The intervention operation robot of claim 37, wherein the second convey assembly includes a first power member, the first power member is located beside the third channel, and the retractable substrate includes a second power member;
the second convey assembly is detachably connected with the retractable substrate, and in response to determining that the second convey assembly is connected with the retractable substrate, the first power member is configured to drive the second intervention instrument to move in the third channel by controlling the third power member to move.

39. The intervention operation robot of claim 25, wherein the instrument conveying member further includes a protective sleeve located at a front end of the instrument conveying member, the protective sleeve is made of a flexible material and configured to protect the intervention instrument.

40. (canceled)

41. The intervention operation robot of claim 25, wherein the intervention operation robot future includes a support platform, the support platform includes:
a first support assembly detachably connected with a medical bed; and
a second support assembly slidably connected with the first support assembly; wherein
the robotic arm is connected to the support platform through the second support assembly.

42. An intervention instrument convey device, configured to clamp at least one intervention instrument and convey the at least one intervention instrument to a target position of a subject, comprising:
one or more first convey assemblies, and each of the one or more first convey assemblies is configured to clamp one of at least one intervention instrument and drive the intervention instrument to move along an axial direction of the first convey assembly and/or drive the intervention instrument to rotate around an axis of the first convey assembly.
